SEQUENCE LISTING (20 sequences)

SEQ ID NO: 1 - protein, 26 aa, Artificial Sequence
  Synthetic Construct
KAQKAQAKQAKQAQKAQKAQAKQAKQ

SEQ ID NO: 2 - protein, 26 aa, Artificial Sequence
  Synthetic Construct
RAQRAQARQARQAQRAQRAQARQARQ

SEQ ID NO: 3 - protein, 26 aa, Artificial Sequence
  Synthetic Construct
GAQGAQAGQAGQAQGAQGAQAGQAGQ

SEQ ID NO: 4 - protein, 15 aa, Artificial Sequence
  Synthetic Construct
KAKAKAKAKAKAKAK

SEQ ID NO: 5 - protein, 29 aa, Artificial Sequence
  Synthetic Construct
KAQAKAQAKAQAKAQAKAQAKAQAKAQAK

SEQ ID NO: 6 - protein, 26 aa, Artificial Sequence
  Synthetic Construct
KAQQAQAKQAQQAQKAQQAQAKQAQQ

SEQ ID NO: 7 - protein, 26 aa, Artificial Sequence
  Synthetic Construct
QAQKAQAQQAKQAQQAQKAQAQQAKQ

SEQ ID NO: 8 - protein, 26 aa, Artificial Sequence
  Synthetic Construct
KAQKAQAKQAKQAQKAQKAQAKQAKQ

SEQ ID NO: 9 - protein, 26 aa, Artificial Sequence
  Synthetic Construct
KTVKTVTKVTKVTVKTVKTVTKVTKV

SEQ ID NO: 10 - protein, 18 aa, Artificial Sequence
  Synthetic Construct
VYKVKTKVKTKVKTKVKT

SEQ ID NO: 11 - protein, 30 aa, Artificial Sequence
  Synthetic Construct
AQAYSKAQKAQAKQAKQAQKAQKAQAKAKQ

SEQ ID NO: 12 - protein, 31 aa, Artificial Sequence
  Synthetic Construct
AQAYARAQRAQARQARQAQRAQRAQARQARQ

SEQ ID NO: 13 - protein, 40 aa, Artificial Sequence
  Synthetic Construct
KAQKAQAKQAKQAQKAQKAQAKQAKQAQKAQKAQAKQAKQ

SEQ ID NO: 14 - protein, 40 aa, Artificial Sequence
  Synthetic Construct
RAQRAQARQARQAQRAQRAQARQARQAQRAQRAQARQARQ

SEQ ID NO: 15 - protein, 27 aa, Artificial Sequence
  Synthetic Construct
  VARIANT 1, 3, 4, 6, 7, 8, 10, 11, 13, 14, 15, 17, 18, 20, 21,22, 24, 25, 27: Xaa = Any amino acid including a modified amino acid that is not charged
  VARIANT 2, 5, 9, 12, 16, 19, 23, 26: Xaa = Any positively charged amino acid
XXXXXXXXXXXXXXXXXXXXXXXXXXX

SEQ ID NO: 16 - protein, 26 aa, Artificial Sequence
  Synthetic Construct
  VARIANT 2, 5, 7, 10, 13, 16, 19, 21, 24: Xaa = Ile, Leu, Met, Val, Gly, Phe, Trp, Tyr, Ser, Thr, Asp, or a modified amino acid that is not charged
  VARIANT 1, 4, 8, 11, 15, 18, 22, 25: Xaa = Arg, Lys, or His
  VARIANT 3, 6, 9, 12, 14, 17, 20, 23, 26: Xaa = Ile, leu, Met, Val, Gly, Phe, Trp, Tyr, Ser, Thr, Asp, or a modified amino acid that is not charged
XXXXXXXXXXXXXXXXXXXXXXXXXX

SEQ ID NO: 17 - protein, 27 aa, Artificial Sequence
  Synthetic Construct
SRAQRAQARQARQAQRAQRAQARQARQ

SEQ ID NO: 18 - protein, 4 aa, Artificial Sequence
  Synthetic Construct
CGGY

SEQ ID NO: 19 - protein, 4 aa, Artificial Sequence
  Synthetic Construct
GGGY

SEQ ID NO: 20 - protein, 31 aa, Artificial Sequence
  Synthetic Construct
CGGYSRAQRAQARQARQAQRAQRAQARQARQ

(19) United States
(12) Patent Application Publication
(10) Pub. No.: US 2024/0003912 A1
(43) Pub. Date: Jan. 4, 2024

(54) METHOD FOR DIAGNOSING AMYLOID DISEASES

(71) Applicant: UNIVERSITY OF TENNESSEE RESEARCH FOUNDATION, Knoxville, TN (US)

(72) Inventors: Jonathan S. WALL, Knoxville, TN (US); Robert Eric HEIDEL, Knoxville, TN (US)

(21) Appl. No.: 18/039,949

(22) PCT Filed: Dec. 3, 2021
(86) PCT No.: PCT/US2021/072729

§ 371 Date: Jun. 1, 2023

Related U.S. Application Data
(60) Provisional application No. 63/121,779, filed on Dec. 4, 2020.

(51) Int. Cl. G01N 33/68

(52) U.S. Cl. CPC G01N 33/6893; G01N 2333/4709

(57) ABSTRACT

Provided herein are methods of detecting and diagnosing types of amyloid-related diseases. Also provided herein are methods of treatment comprising selecting a treatment based upon a particular type of amyloid disease.

### CROSS-REFERENCE TO RELATED APPLICATIONS

This application claims priority to U.S. Provisional Application No. 63/121,779 filed on Dec. 4, 2020, the contents of which are incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 165992000440SEQLIST.TXT, date recorded: Nov. 29, 2021, size: 7,103 bytes).

### FIELD

The present invention relates to methods of detecting and diagnosing types of amyloid-related diseases.

### BACKGROUND

Amyloidosis is a fatal protein-folding disorder characterized by the aggregation and deposition of proteinaceous fibrils and heparan sulfate proteoglycan in vital organs and tissues (Merlini, G. et al. (2003) N. Engl. J. Med. 349, 583-596; Merlini, G. et al. (2004) J. Intern. Med. 255, 159-178; De Lorenzi, E. et al. (2004) Curr. Med. Chem. 11, 1065-1084; Merlini, G. (2004) Neth. J. Med. 62, 104-105). The unrelenting accumulation of amyloid invariably leads to organ dysfunction and severe morbidity or death. The deposits can be cerebral, as in patients with Alzheimer's, Huntington's or prion diseases, or peripheral such as seen in patients with light chain (AL) amyloidosis and type 2 diabetes. Further sub-grouping into localized or systemic indicates whether the precursor protein is produced locally (at the site of deposition) or circulates in the blood stream, respectively (Westermark, P. et al. (2007) Amyloid. 14, 179-183). Amyloid can affect any organ or tissue but the kidneys, pancreas, liver, spleen, nervous tissue and heart constitute the major sites of deposition in patients with familial or sporadic forms of peripheral amyloid disease. Alzheimer's disease currently affects more than 4 million Americans and this figure is estimated to increase to more than 16 million by the year 2050. It is by far the most common form of amyloidosis and poses the greatest socioeconomic impact. In contrast, the peripheral amyloidoses are orphan disorders but account for more than 5,000 new patients annually in the USA alone.

Of these, the major peripheral amyloidosis is AL, a sporadic monoclonal plasma cell dyscrasia resulting in the deposition of fibrils composed of immunoglobulin light chain proteins. AL accounts for approximately two thirds of all peripheral amyloid cases and has a calculated incidence of ˜1.4 per 100,000 persons per year in the USA, which is comparable to that of acute lymphocytic and chronic myeloid leukemias (Group, U.S.C. S. W. (2007) United States Cancer Statistics: 1999-2003 Incidence and Mortality Web-Based Report, U.S. Department of Health and Human Services Centers for Disease Control and Prevention National Cancer Institute, Atlanta). Although AL is one fifth as common as the related plasma cell dyscrasia multiple myeloma it is arguably more devastating with a median survival of only 13.2 months due partly to the rapidly progressive nature of the organ destruction, the lack of effective anti-amyloid therapeutics and the inability to effectively diagnose the disease before organ failure occurs. Fewer than 5% of all AL patients survive 10 years or more from the time of diagnosis (Comenzo, R. L. et al. (2002) Blood 99, 4276-4282). Moreover, in patients with cardiac AL amyloidosis the median survival is less than 5 months. Unfortunately, there is no effective mouse model of AL disease.

ATTR is a form of systemic amyloidosis. 25% of patients with ATTR amyloidosis die within 24 months of diagnosis. (Gertz and Dispenzieri JAMA 324(1)79-89 (2002).) Current therapies do not prevent organ damage. ATTR amyloidosis is caused by transtheryretin (TTR) fibrils. Transthyretin is a protein made by the liver that helps carry thyroid hormone and vitamin A in the blood. Normally, TTR is a tetramer made up of 4 single-chain monomers. In hereditary ATTR amyloidosis, TTR gene mutations are thought to destabilize the protein and cause tetramer dissociation into monomers, which aggregate into amyloid fibrils. In wild-type ATTR amyloidosis, the normal TTR protein becomes unstable, misfolds, and forms amyloid fibrils.

LECT2 amyloidosis (ALECT2) is another common form of amyloidosis, caused by the LECT2 protein. The disorder commonly presents with renal disease that in general is advanced or at an end stage. Associated signs and symptoms of their renal disease may include fatigue, dehydration, blood in urine, and/or other evidence for the presence of the nephrotic syndrome or renal failure. LECT2 amyloidosis causes significant kidney disease in older individuals. It has been suggested that individuals with the disease have an increase in LECT2 production and/or a decrease in LECT2 catabolism. Although mutations in the LECT2 gene have been identified, no mutations have been linked to ALECT2.

In addition to the disorders in which the etiopathology of amyloid is well established, fibrillar deposits with the structural and tinctorial properties of amyloid have been identified in other syndromes although their relevance to the disease state has yet to be established. In type 2 diabetes for example, islet amyloid precursor protein (IAPP) deposits as amyloid in the Islets of Langerhans (Jaikaran, E. T. et al. (2001) Biochim. Biophys. Acta 1537, 179-203). The aggregation of IAPP results in oligomeric structures that are toxic to pancreatic cells (Lin, C. Y. et al. (2007) Diabetes 56, 1324-1332). Thus, it is suggested that the formation of IAPP amyloid in type 1 diabetic patients contributes to R cell destruction and ushers in the transition to insulin dependence (Jaikaran, E. T. et al. (2001) Biochim. Biophys. Acta 1537, 179-203). In another example, plaques containing amyloid fibrils composed of apolipoprotein A-I have been identified in over half of patients with atherosclerotic carotid arteries (Westermark, P. et al. (1995) Am. J. Pathol. 147, 1186-1192; Mucchiano, G. I. et al. (2001) J. Pathol. 193, 270-275). The deposition of these fibrils was more common in older patients but apoA-I is undoubtedly present early in plaque development (Vollmer, E. et al. (1991) Virchows Arch. A. Pathol. Anat. Histopathol. 419, 79-88). As a final example, Apo-A-I amyloid was also recently identified in knee joint menisci obtained from patients having knee replacement surgery and may contribute to the physical deterioration of the joint (Solomon, A. et al. (2006) Arthritis Rheum. 54, 3545-3550).

In total more than 25 proteins have been chemically or serologically identified as constituents of fibrils in amyloid deposits. It is the nature of these proteins that differentiate the diseases, determine the treatment, and establish the prognosis. Although amyloid fibrils are associated with a clinically heterogeneous group of diseases and can form from structurally distinct and functionally diverse precursor proteins, the deposits themselves share a number of remarkably similar characteristics including fibril structure, fibril epitopes and accrual of similar accessory molecules including heparan sulfate proteoglycans (HSPGs). Amyloid is a heterogeneous complex that includes, in addition to fibrils, glycosaminoglycans (GAGs) and in particular the perlecan HSPG (Ancsin, J. B. (2003) Amyloid 10, 67-79; Ailles, L. et al. (1993) Lab. Invest. 69, 443-448; Kisilevsky, R. (1994) Mol. Neurobiol. 9, 23-24; Kisilevsky, R. (1990) Lab. Invest. 63, 589-591; Snow, A. D. et al. (1987) Lab. Invest. 56, 120-123; Li, J. P. et al. (2005) Proc. Natl. Acad. Sci. USA 102, 6473-6477).

In different types of systemic amyloidosis, amyloid deposits can occur in multiple organs such as the kidneys, pancreas, liver, spleen, heart, and nervous tissue, thus making it difficult to differentiate different types of amyloidosis from one another. At the same time, promising therapies targeting treatment of specific types of amyloids are being developed. Thus there is a need for methods to diagnose and differentiate amyloid-related diseases from one another.

### BRIEF SUMMARY OF THE INVENTION

Provided herein are methods for diagnosing a type of amyloid disease. In some embodiments, the method comprises administering an amyloid reactive agent or detection dye. In some embodiments, the method comprises measuring the organ distribution pattern of the amyloid-reactive agent or detection dye in one or more organs. In some embodiments, the organ distribution pattern of the amyloid-reactive agent or detection dye indicates a type of amyloid disease.

Also provided herein are method of treating an amyloid disease. In some embodiments, the method comprises administering an amyloid-reactive agent or detection dye. In some embodiments, the method comprises measuring the organ distribution pattern of the amyloid-reactive agent or detection dye for one or more organs, wherein the organ distribution pattern of the amyloid-reactive agent or detection dye indicates a type of amyloid disease. In some embodiments, the method comprise selecting a treatment based upon the type of amyloid disease. In some embodiments, the method comprises administering the treatment to the individual.

Also provided herein is a method of diagnosing a type of amyloid disease. In some embodiments, the method comprise receiving organ distribution pattern data for an amyloid-reactive-agent or detection dye for an individual. In some embodiments, the method comprises calculating an organ-to-organ ratio for two or more organs, wherein the organ-to-organ ratio is used to diagnose a type of amyloid disease.

In some embodiments, the type of amyloid disease comprises systemic amyloidosis. In some embodiments, type of amyloid disease is selected from the group consisting of amyloid light chain amyloidosis (AL), transthyretin-associated amyloidosis (ATTR), and ALECT2.

In some embodiments, the organ is selected from the group consisting of heart, spleen, kidney, and liver.

In some embodiments, the amyloid-reactive agent is an amyloid-reactive peptide that is detectably labeled. In some embodiments, the amyloid-reactive peptide comprises the amino acid sequence set forth in SEQ ID NO: 1, SEQ ID NO: 2, SEQ ID NO: 13, or SEQ ID NO: 14. In some embodiments, the amyloid-reactive agent reacts with Aβ fibrils. In some embodiments, the amyloid-reactive agent is selected from the group consisting of florbetapir, florbetaben and flutemetamol.

In some embodiments, the detection dye is ThT. In some embodiments, the amyloid-reactive agent reacts with synthetic fibrils composed of light chains or fragments thereof.

In some embodiments, the organ distribution pattern is measured using PET/CT images. In some embodiments, the amyloid-reactive agent is radiolabeled.

In some embodiments, an organ to blood ratio of the amyloid-reactive-agent or detection dye is calculated. In some embodiments, an organ to organ ratio of the amyloid-reactive-agent or detection dye is calculated. In some embodiments, the organ to organ ratio is selected from the group consisting of liver to heart, spleen to heart, spleen to liver, spleen to kidney, kidney to heart, and kidney to liver. In some embodiments a heart to spleen ratio is calculated. In some embodiments, if the heart to spleen ratio is above 1.4, the individual is diagnosed with ATTR amyloidosis.

Also provided herein is a kit for diagnosing a type of amyloid disease comprising an amyloid-reactive agent or detection dye and instructions for use. In some embodiments, the kit is for detecting or diagnosing systemic amyloidosis. In some embodiments, the kit is for detecting or diagnosing amyloid light chain amyloidosis (AL), transthyretin-associated amyloidosis (ATTR), or ALECT2.

In some embodiments, the kit comprises an amyloid-reactive peptide that is detectably labeled. In some embodiments, the amyloid-reactive peptide comprises the amino acid sequence set forth in SEQ ID NO: 1, SEQ ID NO: 2, SEQ ID NO: 13, or SEQ ID NO: 14. In some embodiments, the amyloid-reactive agent reacts with Aβ fibrils. In some embodiments, the amyloid-reactive agent reacts with synthetic fibrils composed of light chains or fragments thereof.

In some embodiments, the kit comprises an amyloid-reactive agent is selected from the group consisting of florbetapir, florbetaben and flutemetamol.

In some embodiments, the kit comprises the detection dye ThT.

In some embodiments, the kid comprises instructions for measuring an organ distribution pattern.

In some embodiments, the kit comprises an amyloid-reactive agent that is radiolabeled.

In some embodiments, the kit comprises instructions for calculating an organ to blood ratio of the amyloid-reactive-agent or detection dye.

In some embodiments, the kit comprises instructions for calculating an organ to organ ratio of the amyloid-reactive-agent or detection dye. In some embodiments, the organ to organ ratio is of liver to heart, spleen to heart, spleen to liver, spleen to kidney, kidney to heart, or kidney to liver.

In some embodiments, the kit provides instructions to provide a diagnosis of ATTR amyloidosis if the heart to spleen ratio is above 1.4.

In some embodiments, the kit comprises instructions to administer the treatment to the individual based upon the diagnosis.

In some embodiments, the kit comprises a therapeutic agent for treating a type of amyloid.

### BRIEF DESCRIPTION OF THE FIGURES

FIG. 1 shows a partial list of amyloid and amyloid related disorders.

FIG. 2A shows organ:blood pool ratios for light chain-associated (AL) amyloidosis (n=14). FIG. 2B shows organ:blood pool ratios for transytherin-associated (ATTR) amyloidosis (n=7). FIG. 2C shows organ:blood pool ratios for leukocyte chemotactic factor 2-associated (ALECT2) amyloidosis (n=2) patients. The organ:blood ratios were calculated from region-of-interest (ROI) analysis of the heart, liver, spleen and left kidney.

FIG. 3 shows the mean organ:blood pool ratios for AL, ATTR, and ALECT2 patients calculated ROI analysis of the heart, liver, spleen and left kidney.

FIG. 4A shows the a summary of the organ:blood pool ratios for AL patients after removal of outlier data points. FIG. 4B shows a summary of the organ:blood pool ratios for ATTR patients after removal of outlier data points. FIG. 4C shows a summary of the organ:blood pool ratios for ALECT2 patients after removal of outlier data points.

FIG. 5 shows the mean organ:blood pool ratios for AL, ATTR, and ALECT2 patients after removal of outlier data points.

FIG. 6 shows the receiver operator characteristic (ROC) analysis performed using single-organ standard uptake value ratio (SUVR) values for ATTR. The ROC curve is shown for heart.

FIGS. 7A-7C show the receiver operator characteristic (ROC) analysis performed using single-organ standard uptake value ratio (SUVR) values for AL. FIGS. 7A, 7B and 7C show the ROC curves for liver, spleen, and kidney, respectively. Diagonal segments are produced by ties.

FIGS. 8A-8F show the receiver operator characteristic (ROC) analysis for detecting ATTR amyloidosis. ROC curves were generated using organ-to-organ uptake ratios for ATTR amyloidosis. FIG. 8A shows the analysis for the heart/spleen ratio. FIG. 8B shows the analysis for the heart/liver ratio. FIG. 8C shows the analysis for the heart/kidney ratio. FIG. 8D shows the analysis for the liver/spleen ratio. FIG. 8E shows the analysis for the liver/kidney ratio. FIG. 8F shows the analysis for the kidney/spleen ratio.

FIGS. 9A-9F show the receiver operator characteristic (ROC) analysis for detecting AL. ROC curves were generated using organ-to-organ uptake ratios for AL. FIG. 9A shows the analysis for the liver/heart ratio. FIG. 9B shows the analysis for the spleen/heart ratio. FIG. 9C shows the analysis for the spleen/liver ratio. FIG. 9D shows the analysis for the spleen/kidney ratio. FIG. 9E shows the analysis for the kidney/heart ratio. FIG. 9F shows the analysis for the kidney/liver ratio.

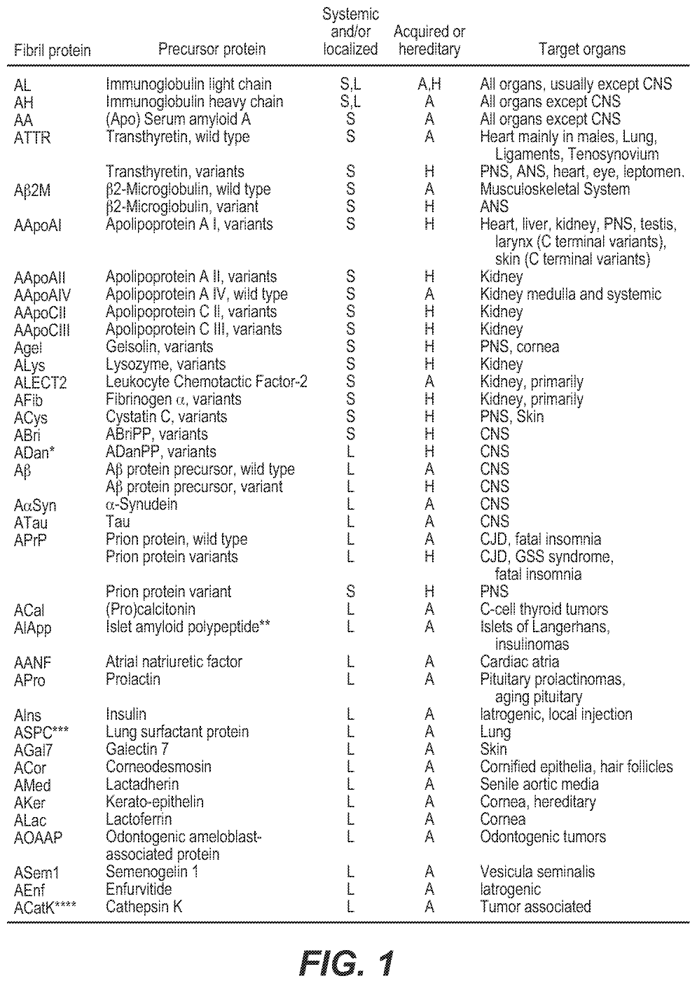

### DETAILED DESCRIPTION

Provided herein are methods for the diagnosis and treatment of amyloid disease. In some embodiments, the methods comprise the administration of an amyloid-reactive agent or detection dye. In some embodiments, the methods further comprise measuring the organ distribution pattern of the amyloid-reactive agent or dye in one or more organs. In some embodiments, the methods provided herein are able to differentiate between different types of systemic amyloidosis such as AL, ATTR, and ALECT2 based upon the organ distribution pattern of an amyloid-reactive agent or detection dye. In some embodiments, the method comprises providing a diagnosis of a type of amyloid disease based upon the organ distribution pattern of an amyloid-reactive agent or detection dye. In some embodiments, the method further comprises selecting a therapy based upon the type of amyloid disease.

As used herein, an “amino acid” or “amino acid residue” refers to any naturally occurring amino acid, any non-naturally occurring amino acid, any modified including derivatized amino acid, or any amino acid mimetic known in the art. The amino acid may be referred by both their common three letter abbreviation and single letter abbreviation.

The terms amyloids, amyloid deposits, amyloid fibrils, and amyloid fibers refer to insoluble fibrous protein aggregates sharing specific structural traits. The protein aggregates have a tertiary structure, for example, that is formed by aggregation of any of several different proteins and that consists of an ordered arrangement of R sheets stacked perpendicular to a fiber axis. See Sunde et al., J. Mol. Biol. (1997) 273:729-39. Abnormal accumulation of amyloids in organs may lead to amyloidosis. Although they are diverse in their occurrence, all amyloids have common morphologic properties in that they stain with specific dyes such as Congo red and have a characteristic red-green birefringent appearance in polarized light after staining. Amyloids also share common ultrastructural features and common x-ray diffraction and infrared spectra.

Amyloidosis refers to a pathological condition or disease characterized by the presence of amyloids, such as the presence of amyloid deposits. “Amyloid diseases” or “amyloidosis” are diseases associated with the formation, deposition, accumulation or persistence of amyloid fibrils. Such diseases include, but are not limited to, Alzheimer's disease, Down's syndrome, hereditary cerebral hemorrhage with amyloidosis of the Dutch type, and cerebral beta-amyloid angiopathy. Other amyloid diseases such as systemic AA amyloidosis, AL amyloidosis, ATTR amyloidosis, ALECT2 amyloidosis, and IAPP amyloidosis of type II diabetes are also amyloid diseases.

As used herein, the term “carriers” includes pharmaceutically acceptable carriers, excipients, or stabilizers which are nontoxic to the cell, tissue, mammal, or subject being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween®, polyethylene glycol (PEG), and Pluronics®.

As used herein, the term “effective amount” or “suitable amount” is an amount sufficient to effect beneficial or desired clinical or biochemical results. An effective amount can be administered one or more times. For purposes of this invention, an effective amount of an amyloid reactive agent or detection is an amount that is sufficient to bind to and allow detection of amyloids.

As used herein, the term “imaging agent” or “contrast agent” which terms may be used interchangeably, refers to any agent which may be used in connection with methods for imaging an internal region of a subject and/or diagnosing the presence or absence of a disease in a subject by the application and/or detection of an energy source. Exemplary imaging agents include contrast agents for use in connection with ultrasound, magnetic resonance imaging, radionuclide imaging, or x-ray (including computed tomography) imaging of a patient, and the compositions described herein.

As used herein, the term “mammal” for purposes of the present invention refers to any animal classified as a mammal, including humans, domestic and farm animals, and zoo, sports, or pet animals, such as dogs, cats, cattle, horses, sheep, pigs, and so on. In some embodiments, the mammal is human.

As used herein, the term “peptide” refers to any peptide or peptidomimetic structure comprising or consisting of two or more amino acids, including chemical modifications and derivatives of amino acids.

As used herein, the term “purified” or “isolated” molecule refers to biological or synthetic molecules that are removed from their natural environment and are isolated or separated and are free from other components with which they are naturally associated.

As used herein, the term “specifically binds” refers to a non-random binding reaction between two molecules, for example between an amyloid-reactive agent or detection dye and an amyloid. The term “specifically binds” may be used interchangeably with “selectively targets” or “selectively associates.”

As used herein, the term “selectively targets” or “selectively associates” with reference to amyloids, refers to, for example, the selective localization or binding between an amyloid-reactive agent or detection dye and an amyloid compared to a non-amyloid protein. An amyloid-reactive agent or detection dye can selectively target multiple types of amyloid.

As used herein, the term “subject” refers to a vertebrate. The vertebrate may be a mammal, for example, a human. The subject may be a human patient.

As used here, the term “amyloid-reactive agent” is an agent that specifically reacts with or binds to amyloid.

### I. Methods of Diagnosis

Some aspects of the present disclosure provide for methods of diagnosing an amyloid disease. In some embodiments, the methods for diagnosing a type of amyloid disease comprise administering an amyloid-reactive agent or detection dye, and measuring the organ distribution pattern of the amyloid-reactive agent or dye in one or more organs. In other embodiments, the methods for diagnosing a type of amyloid disease comprise administering an amyloid-reactive agent or detection dye, and measuring the organ-to-organ ratio of the amyloid-reactive agent or dye in one or more organs. In some embodiments, the organ distribution pattern of the amyloid-reactive agent or dye or the organ-to-organ ratio indicates a type of amyloid disease.

In some embodiments, the methods for diagnosing a type of amyloid disease comprise administering an amyloid-reactive agent or detection dye comprising an amyloid-reactive peptide. In some embodiments, the amyloid-reactive agent or detection dye comprises a peptide, a fusion protein, a small molecule compound, or an antibody or fragment thereof.

In some embodiments, the methods for diagnosing a type of amyloid disease comprise administering an amyloid-reactive agent or detection dye comprising an amyloid-reactive peptide. In some embodiments, the amyloid-reactive peptide comprises an amino acid sequence that is at least 80%, 85%, 90% or more identical to the amino acid sequence set forth as any one of SEQ ID NOS: 1-14, such as at least 95%, at least 96%, at least 97%, at least 98%, at least 99%, or 100% identical to the amino acid sequence set forth as any one of SEQ ID NOS: 1-14. In some embodiments, amyloid-reactive peptides used with the methods described herein comprise or consist of from about 10 to 55 amino acids. The amyloid-reactive peptides of the present invention may, for example, comprise or consist of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 amino acids. Such peptides are described, for example, in international patent application WO2016032949, which is hereby incorporated herein in its entirety. In some embodiments, the methods for diagnosing a type of amyloid disease comprise an amyloid-reactive peptide with an amino acid sequence as set forth in SEQ ID NO: 13. In some embodiments, the methods for diagnosing a type of amyloid disease comprise p5+14.

TABLE 1
Example Amyloid-Reactive Peptide Sequences

| PEP- | | SEQ ID |
|---|---|---|
| TIDE | PRIMARY SEQUENCE: | NO |
| P5 | KAQKA QAKQA KQAQK AQKAQ AKQAK Q | SEQ ID |
| | | NO: 1 |
| P5R | RAQRA QARQA RQAQR AQRAQ ARQAR Q | SEQ ID |
| | | NO: 2 |
| P5G | GAQGA QAGQA GQAQG AQGAQ AGQAG Q | SEQ ID |
| | | NO: 3 |
| P8 | KAKAK AKAKA KAKAK | SEQ ID |
| | | NO: 4 |
| P9 | KAQAK AQAKA QAKAQ AKAQA KAQAK | SEQ ID |
| | AQAK | NO: 5 |
| P19 | KAQQA QAKQA QQAQK AQQAQ AKQAQ Q | SEQ ID |
| | | NO: 6 |
| P20 | QAQKA QAQQA KQAQQ AQKAQ AQQAK Q | SEQ ID |
| | | NO: 7 |
| P31 | KAQKA QAKQA KQAQK AQKAQ AKQAK Q | SEQ ID |
| | | NO: 8 |
| P37 | KTVKT VTKVT KVTVK TVKTV TKVTK V | SEQ ID |
| | | NO: 9 |
| P42 | VYKVK TKVKT KVKTK VKT | SEQ ID |
| | | NO: 10 |
| P43 | AQAYS KAQKA QAKQA KQAQK AQKAQ | SEQ ID |
| | AKAK Q | NO: 11 |
| P44 | AQAYA RAQRA QARQA RQAQR AQRAQ | SEQ ID |
| | ARQAR Q | NO: 12 |
| P5 + | KAQKA QAKQA KQAQK AQKAQ AKQAK | SEQ ID |
| 14 | QAQKA QKAQA KQAKQ | NO: 13 |
| P5R + | RAQRA QARQA RQAQR AQRAQ ARQAR | SEQ ID |
| 14 | QAQRA QRAQA RQARQ | NO: 14 |

The amino acids forming all or a part of the amyloid-reactive peptides used with the present methods may be stereoisomers and modifications of naturally occurring amino acids, non-naturally occurring amino acids, post-translationally modified amino acids, enzymatically synthesized amino acids, derivatized amino acids, constructs or structures designed to mimic amino acids, and the like. The amino acids forming the peptides of the present invention may be one or more of the 20 common amino acids found in naturally occurring proteins, or one or more of the modified and unusual amino acids. The amyloid-reactive peptides used with the methods described herein may be made by any technique known to those of skill in the art, including chemical synthesis or recombinant means using standard molecular biological techniques.

The peptides of the present invention may also comprise one or more modified amino acids. The modified amino acid may be a derivatized amino acid or a modified and unusual amino acid. Examples of modified and unusual amino acids include but are not limited to, 2-Aminoadipic acid (Aad), 3-Aminoadipic acid (Baad), β-Amino-propionic acid (Bala, β-alanine), 2-Aminobutyric acid (Abu, piperidinic acid), 4-Aminobutyric acid (4Abu), 6-Aminocaproic acid (Acp), 2-Aminoheptanoic acid (Ahe), 2-Aminoisobutyric acid (Aib), 3-Aminoisobutyric acid (Baib), 2-Aminopimelic acid (Apm), 2,4-Diaminobutyric acid (Dbu), Desmosine (Des), 2,2′-Diaminopimelic acid (Dpm), 2,3-Diaminopropionic acid (Dpr), N-Ethylglycine (EtGly), N-Ethylasparagine (EtAsn), Hydroxylysine (Hyl), allo-Hydroxylysine (AHyl), 3-Hydroxyproline (3Hyp), 4-Hydroxyproline (4Hyp), Isodesmosine (Ide), allo-Isoleucine (Alle), N-Methylglycine (MeGly, sarcosine), N-Methylisoleucine (Melle), 6-N-Methyllysine (MeLys), N-Methylvaline (MeVal), Norvaline (Nva), Norleucine (Nle), and Ornithine (Orn).

Other examples of modified and unusual amino acids are described generally in Synthetic Peptides: A User's Guide, Second Edition, April 2002, Edited Gregory A. Grant, Oxford University Press; Hruby V J, Al-obeidi F and Kazmierski W: Biochem J 268:249-262, 1990; and Toniolo C: Int J Peptide Protein Res 35:287-300, 1990; the teachings of all of which are incorporated herein by reference.

The peptides of the present may comprise at least about 15% positively charged amino acids such as arginine and/or lysine. The peptides comprise from about 15% to about 50%, about 20% to about 45%, about 25% to about 40%, or about 30% to about 35% positively charged amino acids. In one embodiment, the peptides of the present invention may comprise the following amino acid sequence:

(SEQ ID NO: 15)
XBXXBXXXBXXBXXXBXXBXXXBXXBX,

wherein

X is any amino acid including a modified amino acid that is not charged; and, B is a positively charged amino acid.

In one embodiment, the peptides of the present invention comprises SEQ ID NO: 15, wherein X is alanine, valine, serine, threonine, or glycine and B is arginine, lysine, or histidine. The peptides of the present invention may comprise or consist of SEQ ID NO: 15. The peptides of the present invention may have at most 55 amino acids and comprise the amino acid sequence as set forth in SEQ ID NO: 15.

In another embodiment, the peptide may comprise the following amino acid sequence: BXZBXZXBZXBZXZBXZBXZXBZXBZ, (SEQ ID NO: 16) wherein, B is arginine, lysine, or histidine; X is isoleucine, leucine, methionine, valine, glycine, phenylalanine, tryptophan, tyrosine, serine, threonine, asparagine, or a modified amino acid that is not charged; and Z may be isoleucine, leucine, methionine, valine, glycine, phenylalanine, tryptophan, tyrosine, serine, threonine, asparagine, or a modified amino acid that is not charged. In certain embodiments, the peptides of the present invention may comprise or consist of the following amino acid sequence: SRAQRAQARQARQAQRAQRAQARQARQ. (SEQ ID NO: 17)

The peptides of the present invention may be a fusion protein comprising a second peptide as a leader sequence at the amino terminus, such as CGGY (SEQ ID NO: 18) or GGGY (SEQ ID NO: 19) for labeling with an agent for detection. Accordingly, in some embodiments, the amyloid-reactive agent may have at most 55 amino acids and comprise an amino acid sequence as set forth in SEQ ID NO: 17. CGGYSRAQRAQARQARQAQRAQRAQARQARQ. (SEQ ID NO: 20)

The fusion protein may comprise other leader sequences such as a cell penetrating peptide (CPP) or a blood brain barrier (BBB) translocating peptide.

The present invention also provides other peptides and fusion proteins that are rich in positively charged amino acids for imaging amyloids.

The peptides of the present invention may be made by any technique known to those of skill in the art, including chemical synthesis, recombinant means using standard molecular biological techniques, or the isolation of peptides from natural sources. The peptides may be synthesized in solution or on a solid support in accordance with conventional techniques. Various automatic synthesizers are commercially available and can be used in accordance with known protocols. (See, for example, Stewart and Young, Solid Phase Peptide Synthesis, 2d ed. Pierce Chemical Co., 1984; Tam et al., J. Am. Chem. Soc., 105:6442, 1983; Merrifield, Science, 232: 341-347, 1986; and Barany and Merrifield, The Peptides, Gross and Meienhofer, eds., Academic Press, New York, pp. 1-284, 1979, each is incorporated herein by reference in its entirety.)

Alternatively, recombinant DNA technology may be employed wherein a nucleotide sequence which encodes a peptide of the invention is inserted into an expression vector, transformed or transfected into an appropriate host cell, cultivated under conditions suitable for expression, and isolating the peptide.

In certain embodiments, amyloid reactive agent may be a naturally occurring peptide and may be obtained by isolation or purification from its natural sources. Protein purification techniques involve, at one level, the homogenization and crude fractionation of the cells, tissue or organ to peptide and non-peptide fractions. Other protein purification techniques include, for example, precipitation with ammonium sulfate, polyethylene glycol (PEG), antibodies and the like, or by heat denaturation, followed by: centrifugation; chromatography steps such as ion exchange, gel filtration, reverse phase, hydroxylapatite and affinity chromatography; isoelectric focusing; gel electrophoresis, for example polyacrylamide gel electrophoresis; and combinations of these and other techniques

Various chromatographic techniques include but are not limited to ion-exchange chromatography, gel exclusion chromatography, affinity chromatography, immunoaffinity chromatography, and reverse phase chromatography. A particularly efficient method of purifying peptides is fast performance liquid chromatography (FPLC) or even high performance liquid chromatography (HPLC).

The order of conducting the various purification steps may be changed, or that certain steps may be omitted, and still result in a suitable method for the preparation of a substantially purified peptide. The peptides of the present invention may be a part of a polypeptide or protein and may be produced by biochemical or enzymatic fragmentation of the polypeptide or protein. Accordingly, the peptides of the present invention may be (a) naturally-occurring, (b) produced by chemical synthesis, (c) produced by recombinant DNA technology, (d) produced by biochemical or enzymatic fragmentation of larger molecules, (e) produced by methods resulting from a combination of methods a through d listed above, or (f) produced by any other means for producing peptides.

During chemical synthesis, the peptides may be modified at its N- or C-terminus, thereby providing for improved stability and formulation, resistance to protease degradation, and the like. Examples of modifications of amino acids include pegylation, acetylation, alkylation, formylation, amidation. Moreover, various amino acids which do not naturally occur along the chain may be introduced to improve the stability of the peptides.

Cysteine is also useful for facilitating the labeling of peptides of the present invention with biotin, fluorophores, or other ligands via conjugation. Moreover, a cysteine on the leader peptide allows the generation of covalently bound dimer molecules that might increase the relative affinity of the peptides for their targets.

In particular embodiments, the methods comprise administering an amyloid-reactive agent or detection dye comprising florbetapir (¹⁸F-florbetapir, Amyvid®), flortaben (¹⁸F-florbetaben, Neuraceq®), or flutementanol (¹⁸F-flutemetamol, Vizamyl®). In another embodiment, the methods for diagnosing the type of amyloid comprise administering an amyloid-reactive agent or detection dye comprising thioflavin T (ThT). Other exemplary amyloid-reactive agents or dyes that may be used with the methods described herein include, without limitation, 3,3-diphosphono-1,2-propanodicarboxylic acid (DPD), hydroxyl-diphosphonate (HDP), hydroxymethylene-diphosphonate (HMDP), stannous pyrophosphate (PyP), NAV4694 (¹⁸F-NAV4694, ¹⁸F-AZD4694), thioflavin S (ThS), a serum amyloid P (SAP) protein or peptide, a serum amyloid A (SAA) protein or peptide, a tau protein or peptide, Congo Red, Congo Corinth, benzopurpurin 4B, Vital Red, Trypan Blue, Amidoblack 10B, Acid Fuchsin, ¹¹C-Pittsburgh Compound B (¹¹C-PIB), ¹⁸F-THK5317, ¹⁸F-THK5351, ¹⁸F-flortaucipir (¹¹⁸F-flortaucipir, ¹⁸F-AV-1451), ¹⁸F-T807, ¹⁸F-fluorodeoxyglucose (¹⁸F-2-fluoro-2-deoxy-D-glucose, ¹⁸F-FDG), ¹⁸F-MK-6240, ¹⁸F-PI-2620, ¹¹C-UCB-J, a mitochondrial translocator protein (TSPO) or peptide, ¹¹C-R-PK11195, ¹⁸F-DPA-714 and ¹¹C-PBR28, ¹¹C-BF-227, or an amyloid-reactive antibody or fragment.

In some embodiments, the method for diagnosing a type of amyloid disease comprises administering an amyloid-reactive agent or detection dye comprising a detectable label. Without being limited, this may include radionuclides (e.g., C-¹¹, I-¹²⁵ I-¹²³, I-¹³¹, Zr-⁸⁹, Tc-^{99m}, Cu-⁶⁴, Br-⁷⁶, F-¹⁸); enzymes (horse radish peroxidase); biotin; fluorophores, etc. Any means known in the art for detectably labeling a protein can be used and/or adapted for use with the methods described herein. For example, the amyloid-reactive peptides, can be radiolabeled with a radioisotope, or labeled with a fluorescent tag or a chemiluminescent tag. Example radioisotopes include, for example, ¹¹C, ¹⁸F, ¹¹¹In, ^{99m}Tc, and ¹²³I, ¹²⁴I, and ¹²⁵I. These and other radioisotopes can be incorporated to the amyloid-reactive agent or detection dye. Example fluorescent or chemiluminescent tags include fluorescein, Texas red, rhodamine, Alexa dyes, and luciferase that can be incorporated to the amyloid-reactive agent or detection dye using conventional methods in the art.

In some embodiments, the methods for diagnosing a type of amyloid disease comprise administering an amyloid-reactive agent or detection dye comprising a radiolabel. In some embodiments, the radiolabel is ¹¹C, ¹⁸F, ¹¹¹In, ^{99m}Tc, ⁸⁹Zr and ¹²³I, ¹²⁴I, or ¹²⁵I. In some embodiments, the radiolabelled amyloid-reactive agent or detection dye is a radiolabeled amyloid-reactive peptide. In some embodiments, the radiolabeled amyloid-reactive peptide is a ¹²⁴I-labelled amyloid-reactive peptide. In other embodiments, the method for diagnosing a type of amyloid disease comprise administering ¹²⁴I-p5+14. In other embodiments, the radiolabeled amyloid-reactive agent or detection dye is florbetapir, flortaben, or flutemetamol.

In another embodiment, the methods for diagnosing a type of amyloid disease comprise administering an amyloid-reactive agent or detection dye comprising a fluorescent label. In some embodiments, the agent fluorescently-labelled amyloid-reactive agent or detection dye is thioflavin T (ThT).

In some embodiments, the amyloid-reactive agent or detection dye comprises an amyloid-reactive peptide conjugated to a radiolabel. In some embodiments, the amyloid-reactive agent or detection dye comprises a peptide conjugated to a bulking agent. In some embodiments, the amyloid-reactive peptide is conjugated to PEG. In some embodiments, the amyloid-reactive peptide is conjugated to an antibody.

In some embodiments, the amyloid-reactive agent or detection dye specifically binds to amyloid deposits. In some embodiments, the amyloid-reactive agent or detection dye is able to detect the presence, absence, or amount of amyloid in the subject. In some embodiments, the amyloid-reactive agent or dye cross-reacts to amyloid deposits formed by a number of different proteins. In some embodiments, the amyloid-reactive agent or detection dye binds to amyloid deposits formed by a variety of proteins and/or peptides. In some embodiments, the amyloid-reactive agent or detection dye binds to amyloid deposits formed by amyloid light chain (AL).

In some embodiments, the amyloid-reactive agent or detection dye binds to amyloid formed by transthyretin (TTR) fibrils. In some embodiments, the amyloid-reactive agent or detection dye binds to amyloid formed by serum amyloid protein A (sAA). In some embodiments, the amyloid-reactive agent or detection dye binds to amyloid formed by amyloidogenic forms of immunoglobulin heavy chain (AH), β₂-microglobulin (Aβ₂M), transthyretin variants (ATTR), apolipoprotein AI (AApoAI), apolipoprotein AII (AApoAII), gelsolin (AGel), lysozyme (ALys), leukocyte chemotactic factor (ALECT2), fibrinogen a variants (AFib), cystatin variants (ACys), calcitonin ((ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), IAAP, ALκ4, or Alλ1.

In some embodiments, the amyloid-reactive agent or detection dye binds to heperan sulfate glycosaminoglycans (GAGs). In some embodiments GAGs are associated with amyloid deposits. Binding of GAGs to amyloid fibrils occurs mainly through electrostatic interactions involving the negative polyelectrolyte charges and positively charged side chains residues of aggregating protein. Similarly to catalyst for reactions, GAGs favor aggregation, nucleation and amyloid fibril formation functioning as a structural templates for the self-assembly of highly cytotoxic oligomeric precursors, rich in β-sheets, into amyloid fibrils. Moreover, the GAGs amyloid promoting activity can be facilitated through specific interactions via consensus binding sites between amyloid polypeptide and GAG molecules.

In some embodiments, the method comprises administering an amyloid-reactive agent or detection dye to an individual. In some embodiments, the amyloid-reactive agent or detection dye is administered in a pharmaceutical composition. In some embodiments, the composition comprises an aqueous buffer. The compositions may also include a solubilizing agent and a local anesthetic such as lignocaine to ease pain at the site of the injection. The ingredients are supplied either separately or mixed together in unit dosage form, for example, as a dry lyophilized powder or water free concentrate in a hermetically sealed container such as an ampule indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

The compositions may further comprise a carrier. The present invention also provides pharmaceutical compositions comprising one or more peptides and/or fusion peptides of the present invention. Such pharmaceutical compositions comprise an effective amount of the peptide or fusion peptide for binding to and detection of amyloids and a pharmaceutically acceptable carrier.

Pharmaceutically acceptable carriers include solid or liquid carriers or components which may be added to enhance or stabilize the composition, or to facilitate preparation of the composition include, without limitation, syrup, water, isotonic saline solution, 5% dextrose in water or buffered sodium or ammonium acetate solution, oils, glycerin, alcohols, among others. Examples of oils include those of petroleum, animal, vegetable or synthetic origin, such as peanut oil, soybean oil, mineral oil, and sesame oil. The carrier may also include a sustained release material such as glyceryl monostearate or glyceryl distearate, alone or with a wax. Other suitable pharmaceutical carriers include but are not limited to include starch, glucose, lactose, sucrose, gelatin, malt, rice, flour, chalk, silica gel, sodium stearate, glycerol monostearate, talc, sodium chloride, dried skim milk, propylene, glycol, water, ethanol, flavoring agents, preservatives, coloring agents diluents, granulating agents, lubricants, binders, and the like.

Water may be the preferred carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents. Such compositions can take the form of solutions, suspensions, emulsion, tablets, pills, capsules, powders, sustained-release formulations and the like. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of other suitable pharmaceutical carriers are described in “Remington's Pharmaceutical Sciences” by E. W. Martin.

Methods for imaging amyloids include but are not limited to magnetic resonance imaging (MRI), computed axial tomography (CAT) scanning, positron emission tomography (PET), ultrasonic imaging, x-rays, radionuclide imaging, single photon emission computed tomography (SPECT), and multiphoton microscopy.

To increase the sensitivity of scans, various contrast media may be used. The contrast media for scans may include all molecules that attenuate x-rays. For positron emission tomography and radionuclide imaging, radioisotopes may be used. All positron emitting isotopes are useful for positron emission tomography radionuclide imaging, and all γ-photon emitting isotopes are useful for radionuclide imaging.

Contrast agents for ultrasonic imaging include positive agents and negative agents. Positive agents reflect the ultrasonic energy and thus they produce a positive (light) image. Correspondingly, negative agents enhance transmissibility or sonolucency and thus produce a negative (dark) image. A variety of substances-gases, liquids, solids, and combinations of these—has been investigated as potential contrast-enhancing agents. Examples of solid particle contrast agents disclosed in U.S. Pat. No. 5,558,854 include but not limited to IDE particles and SHU454. European Patent Application 0231091 discloses emulsions of oil in water containing highly fluorinated organic compounds for providing enhanced contrast in an ultrasound image. Emulsions containing perfluorooctyl bromide (PFOB) have also been examined as ultrasound imaging agents. U.S. Pat. No. 4,900,540 describes the use of phospholipid-based liposomes containing a gas or gas precursor as a contrast-enhancing agent.

Several classes of compounds have potential as MRI contrast agents. These classes include supraparamagnetic iron oxide particles, nitroxides, and paramagnetic metal chelates (Mann et al., 1995). A strong paramagnetic metal is preferred. Normally, paramagnetic lanthanides and transition metal ions are toxic in vivo. Thus, it is necessary to incorporate these compounds into chelates with organic ligands. The peptides and fusion peptides of the present invention may be used to enhance the targeting of such chelated metals to amyloids, which allows for the reduction in the total dose of imaging composition otherwise required.

Imaging agents may be attached to peptides and fusion peptides using known methods. Certain attachment methods involve the use of a metal chelate complex employing, for example, an organic chelating agent such a DTPA. Acceptable chelates are known in the field. They include but are not limited to 1,4,7,10-tetraazacyclododecane-N,N′,N″,N′″-tetraacetic acid (DOTA); 1,4,7,10-tetraazacyclododecane-N,N′,N″-triacetic acid (DO3A); 1,4,7-tris(carboxymethyl)-10-(2-hydroxypropyl)-1,4,7,10-tetraazacyclododecane (HP-DO3A); diethylenetriaminepentaacetic acid (DTPA); and many others.

Several classes of compounds have potential as MRI contrast agents. These classes include supraparamagnetic iron oxide particles, nitroxides, and paramagnetic metal chelates (Mann et al., 1995). A strong paramagnetic metal is preferred. Normally, paramagnetic lanthanides and transition metal ions are toxic in vivo. Thus, it is necessary to incorporate these compounds into chelates with organic ligands. The peptides and fusion peptides of the present invention may be used to enhance the targeting of such chelated metals to amyloids, which allows for the reduction in the total dose of imaging composition otherwise required.

Paramagnetic metals of a wide range are suitable for chelation. Suitable metals include those having atomic numbers of 22-29 (inclusive), 42, 44 and 58-70 (inclusive), and having oxidation states of 2 or 3. Examples of such metals include but are not limited to chromium (III), manganese (II), iron (II), cobalt (II), nickel (II), copper (II), praseodymium (III), neodymium (III), samarium (III), gadolinium (III), terbium (III), dysprosium (III), holmium (III), erbium (III), ytterbium (III), and vanadium (II). Ions useful in other contexts, such as X-ray imaging, include but are not limited to lanthanum (III), gold (III), lead (II), and especially bismuth (III).

Among the radioisotopes that can be used to label peptides and fusion peptides of the present invention that are suitable for localization studies are gamma-emitters, positron-emitters, X-ray-emitters and fluorescence-emitters. Appropriate radioisotopes for labeling peptides and fusion proteins include astatine²¹¹, bromine⁷⁶, ¹⁴carbon, ¹¹carbon, ⁵¹chromium, ³⁶chlorine, ⁵⁷cobalt, ⁵⁸cobalt, copper⁶⁷, copper⁶⁴, ¹⁵²europium, fluorine¹⁸, gallium⁶⁷, Gallium⁶⁸, ³hydrogen, iodine¹²³, iodine¹²⁴, iodine¹²⁵, iodine¹²⁶, iodine¹³¹, indium¹¹¹, indium^{113m}, ⁵⁹iron, ¹⁷⁷lutetium, mercury¹⁰⁷, mercury²⁰³, ³²phosphorus, rhenium¹⁸⁶, rhenium¹⁸⁸, ruthenium⁹⁵, ruthenium⁹⁷, ruthenium¹⁰³, ruthenium¹⁰⁵, rhenium^{99m}, rhenium¹⁰⁵, rhenium¹⁰¹, ⁷⁵selenium, ³⁵sulphur, technitium^{99m}, tellurium^{121m}tellurium^{122m}, tellurium^{125m}, thulium¹⁶⁵, thulium¹⁶⁷, thulium¹⁶⁸, and yttrium⁹⁰. The halogens may be used more or less interchangeably as labels. The gamma-emitters, iodine¹²³ and technetium^{99m}, may also be used because such radiometals are detectable with a gamma camera and have favorable half lives for imaging in vivo. The positron-emitters ¹⁸-fluorine or ¹²⁴iodine which are suitable for PET imaging and have suitable half lives for peptide imaging may also be used. Peptides and fusion peptides of the present invention may be labeled with indium¹¹¹ or technetium^{99m} via a conjugated metal chelator, such as DTPA (diethlenetriaminepentaacetic acid) or covalently and directly to the flanking peptide that contains a Cys residue.

Radioactively labeled peptides or fusion peptides may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Peptides or fusion peptides according to the invention may be labeled with technetium^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent, such as SnCl₂, a buffer solution such as sodium-potassium phthalate solution, and the peptide. Intermediary functional groups that are often used to bind radioisotopes that exist as metallic ions to peptides are diethylenetriaminepenta-acetic acid (DTPA) and ethylene diaminetetra-acetic acid (EDTA), as mentioned earlier.

Other useful labels include fluorescent labels, chromogenic labels, and biotin labels. Fluorescent labels, include but are not limited to rhodamine, fluorescein isothiocyanate, fluorescein sodium, renographin, and Texas Red sulfonyl chloride. In certain embodiments, the peptides and fusion peptides of the present invention may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Secondary binding ligands include biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

The present invention provides a method for detecting amyloids in a subject. The method comprises administering a pharmaceutical composition comprising an effective amount of one or more peptides or fusion peptides of the present invention to a subject and detecting the peptides or fusion peptides bound to the amyloids. The peptides may be labeled with an imaging agent, such as a radioisotope. The peptide has specific binding affinity for the deposits and the binding is detectable. The binding of the peptides or fusion peptides to the amyloids may be detected by MRI, CAT scan, PET imaging, ultrasound imaging, SPECT imaging, X-ray imaging, fluorescence imaging, or radionuclide imaging.

In some instances, the methods for diagnosing a type of amyloid disease comprise administering to an individual a detectable amount of an amyloid-reactive reagent or dye. The detectable amount to be administered may be based on the type of detection to be performed. For example, in some embodiments, a detectable amount of an amyloid-reactive reagent or dye may be an amount sufficient to be detectable by imaging when administered to a subject. The detectable amount of the amyloid-reactive agent or detection dye to be administered to an individual may vary depending upon such factors as the age, sex and weight of the individual, the specific response of the individual, the dosimetry, the formulation, and instrument-related factors. Optimization of such factors is well within the level of skill in the art. The detectable amount of the amyloid-reactive agent or detection dye may also vary with the mode of administration of the amyloid-reactive agent or detection dye.

In some instances, the amyloid-reactive agent or detection dye is administered parenterally, paracancerally, transmucosally, tansdermally, intramuscularly, intravenously, intradermally, subcutaneously, intraperitonealy, intraventricularly, or intracranially. In some instances, the amyloid-reactive agent or detection dye is administered intravenously. In other instances, the amyloid-reactive agent or detection dye is administered intraperitonealy.

One of ordinary skill in the art will further appreciate that an effective amount of the amyloid-reactive agent or detection dye can be administered in a single dose, or can be achieved by administering multiple doses. In some instances, the administration of the amyloid-reactive agent or detection dye may further comprise administering a flushing solution. For example, a flushing solution, e.g. saline, may be administered after immediately after administration of the amyloid-reactive agent or detection dye, or after a set period of time after administration of the amyloid-reactive agent or detection dye. In other instances, the amyloid-reactive agent or detection dye may be metabolized and excreted a certain period of time after administration.

In some embodiments, the methods of diagnosing a type of amyloid disease comprise detecting amyloids with an amyloid-reactive agent or detection dye. Examples of amyloids that can be detected as part of the present methods include, but are not limited to, amyloidogenic forms of immunoglobulin heavy chain (AH), β₂-microglobulin (Aβ₂M), transthyretin variants (ATTR), amyloid beta (Aβ), apolipoprotein AI (AApoAI), apolipoprotein AII (AApoAII), gelsolin (AGel), lysozyme (ALys), leukocyte chemotactic factor (ALect2), fibrinogen a variants (AFib), cystatin variants (ACys), calcitonin (ACal), lactadherin (AMed), islet amyloid polypeptide (AIAPP), prolactin (APro), insulin (AIns), prior protein (APrP); α-synuclein (AαSyn), tau (ATau), atrial natriuretic factor (AANF), or IAAP, and other amyloidogenic peptides. In some embodiments of the present disclosure, the method for diagnosing a type of amyloid disease comprises detecting ATTR, AL and/or ALECT2 amyloids. In other embodiments, the method for diagnosing a type of amyloid disease comprises distinguishing between ATTR, AL and ALect2 amyloids.

In some embodiments, the methods for diagnosing a type of amyloid disease comprise measuring the organ distribution pattern of the amyloid-reactive agent or detection dye, wherein the organ distribution pattern of the amyloid-reactive agent or detection dye indicates a type of amyloid disease. The type of amyloid disease may be a sporadic amyloidosis, or have a genetic component, e.g. hereditary amyloidosis. Some non-limiting examples of amyloid diseases are AA amyloidosis, AL amyloidosis, AH amyloidosis, Aβ amyloidosis, ATTR amyloidosis, ALect2 amyloidosis, and IAPP amyloidosis of type II diabetes, Alzheimer's disease, Down's syndrome, hereditary cerebral hemorrhage with amyloidosis of the Dutch type, cerebral beta-amyloid angiopathy, spongiform encelohalopathy, thyroid tumors, Parkinson's disease, dementia with Lewis bodies, a tauopathy, Huntington's disease, senile systemic amyloidosis, familial hemodialysis, senile systemic aging, aging pituitary disorder, iatrogenic syndrome, spongiform encephalopathies, reactive chronic inflammation, thyroid tumors, myeloma or other forms of cancer. In some embodiments, the type of amyloid disease is a systemic amyloid disease. In some embodiments, the type of amyloid disease is AL amyloidosis, ATTR amyloidosis, or ALECT2 amyloidosis.

In some embodiments, the methods for diagnosing the type of amyloid disease comprise measuring the organ distribution pattern of the amyloid-reactive agent or detection dye in one or more organs. Without being bound by theory, it is thought that the anatomic distribution of amyloid in each of form of the disease may have a specific pattern. For example, the amyloid deposits in ATTR amyloidosis are prevalent in the heart and peripheral nerves, while AL amyloidosis, another common amyloidosis, exhibits a variable pattern of amyloid deposition, with amyloids observed in, for example, the heart, spleen, liver, kidneys, peripheral nerves, gastrointestinal tract, muscle, lung, and lymph nodes. In some embodiments, the methods for diagnosing the type of amyloid disease comprise measuring the organ distribution pattern of the amyloid-reactive agent or detection dye in one or more of heart, spleen, liver, kidneys, peripheral nerves, the gastrointestinal tract, muscle, lungs, brain, and lymph nodes. In some embodiments, the one or more organs are abdominothoracic organs. In some embodiments, the one or more organs are heart, spleen, liver, or kidney.

In some embodiments, the step of measuring the organ distribution pattern of the amyloid-reactive agent or detection dye in one or more organ as in the present methods comprises determining an organ uptake value for each organ. Organ uptake may be determined by methods known to those skilled in the art. For example, the organ uptake value may indicate the relative or absolute levels of the amyloid-reactive agent or detection dye detected in each organ in an individual. In some embodiments, the organ uptake value ratio is a relative uptake value. In some embodiments, the organ uptake value is a standard uptake value for each organ. As would be appreciated by those skilled in the art, the standard uptake value may be determined by measuring the amount of amyloid-reactive agent or detection dye detected in a reactive organ, e.g. heart, relative to the amount of amyloid-reactive agent or detection dye detected in a non-reactive tissue or sample, e.g. blood. The amount of amyloid-reactive agent or detection dye in an organ may be determined, for example, by quantifying the detectable signal from the amyloid-reactive agent or detection dye in an organ, e.g. by computing pixel values in an image. In some embodiments, the standard uptake value is determined as the ratio of the amount of amyloid-reactive agent or detection dye detected in an organ, and the amount of amyloid-reactive agent or detection dye detected in blood. In some embodiments, the organ uptake value is indicative of the organ distribution pattern of the amyloid-reactive agent or detection dye.

In other instances, the methods for determining type of amyloid disease comprise administering an amyloid-reactive agent or detection dye and calculating an organ-to-organ ratio for two or more organs. In some instances, the step of calculating an organ-to-organ ratio for two or more organs comprises calculating the ratio between the organ uptake value for a first organ and the organ uptake value for a second organ. In some instances, the organ-to-organ ration is selected from the group consisting of liver-to-heart, spleen-to-heart, spleen-to-liver, spleen-to-kidney, kidney-to-heart, and kidney-to-liver. In some instances, the organ-to-organ ratio is the heart-to-spleen ratio. In some instances, the organ-to-organ ratio is between 0 and 1, 1, or higher than 1. In some instances, the organ-to-organ is indicative of the type of amyloid disease in an individual.

In some embodiments, an organ uptake value or organ-to-organ ratio are indicative of the type of amyloid disease. In some embodiments, the organ uptake value or the organ-to-organ ratio are indicative of the type of amyloid disease only if they are above a cut-off or threshold value. For example, in some embodiments, if the organ-to-organ ration is 1.4 for a type of amyloid disease, then a diagnosis of that type of amyloid disease will be made if an organ-to-organ ratio of 1.4 or more is calculated for an individual. As another non-limiting example, if an organ uptake value cut-off is 1.4 for a type of amyloid disease, then a diagnosis of that type of amyloid disease is not appropriate if an organ-to-organ ratio below 1.4 is calculated for an individual. The particular cut-off or threshold value for diagnosing the type of amyloid disease may vary with the type of amyloid disease, disease progression, patient demographics, the amyloid-reactive agent or detection dye administered, and the detection method used. In some embodiments, the organ uptake value or organ-to-organ cut-off or threshold value is calculated from data from organ distribution of an amyloid-reactive agent or detection dye. In some embodiments, the organ uptake value or organ-to-organ cut-off or threshold value is calculated from data from a population with a particular type of amyloid disease.

Preferably, the organ uptake values or the organ-to-organ uptake ratio cut-off or threshold values for diagnosing the type of amyloid disease is determined using a receiver operator characteristic curve. As is understood in the art, the receiver operating characteristic curve, or, ROC curve, is a plot of the performance of a particular feature for distinguishing two populations, patients with an amyloid disease, and controls, e.g., those without an amyloid disease. Data across the entire population (namely, the patients and controls) are sorted in ascending order based on the value of a single feature (e.g. organ uptake value). Then, for each value for that feature, the true positive and false positive rates for the data are determined. The true positive rate (sensitivity) is determined by counting the number of cases above the value for that feature under consideration and then dividing by the total number of patients. The false positive rate (specificity) is determined by counting the number of controls above the value for that feature under consideration and then dividing by the total number of controls.

ROC curves can be produced for a single feature as well as for other single outputs, for example, combinations of two or more features are mathematically added together (added, drawn, multiplied, etc.) to provide a single total value, which can be plotted in the ROC curve. Furthermore, any combination of multiple features by which the combination leads to a single output value can be plotted in the ROC curve. These combinations of features may include testing. The ROC curve is a plot of the true positive rate (sensitivity) of the test against the false positive rate (1-specificity) of the test. The area under the ROC curve can be a figure of merit for a given sample population, with the test ranging from 1 to 0 for a complete test that gives a completely random response in classifying the test subjects. As with any diagnostic application, the area under the ROC curve is indicative of the predictive power of the model, and can be used to compare the predictive power of one model against another. Using the ROC curve a cut-off value can be selected for diagnosing an amyloid disease and/or amyloid type in an individual with high confidence.

In some embodiments, and the steps of measuring the organ distribution pattern of the amyloid-reactive agent or detection dye in one or more organs, or the step of calculating the organ-to-organ ratio of amyloid-reactive agent or detection dye comprises analyzing imaging data. The imaging data may be generated by any procedure known in the art that may allow the imaging of the amyloid-reactive reagent or dye. For example, the amyloid-reactive agent or detection dye may be detected by positron emission tomography (PET), computed tomography (CT), magnetic resonance imaging (MRI), or single-photon emission computed tomography (SPECT). In certain embodiments, the amyloid-reactive agent or detection dye may be detected by combined imaging methods such as PET/CT (PET with concurrent computed tomography imaging) or PET/MRI (PET with concurrent magnetic resonance imaging). The imaging procedure may result in one or more images of the region of observation of the individual. In certain embodiments, the imaging results in more than one image, these multiple images may be combined, overlaid, added, subtracted, color coded or otherwise fused and mathematically manipulated by any method known in the art. The image produced may be a digital or analog image that may be displayed as a “hard” image on, for example, printer paper, photographic paper or film, or as an image on a screen, such as for example, a video or LCD screen.

In some embodiments, PET images are analyzed using a region of interest (ROI) method. In some embodiments, the images are planar images. In some embodiments, the images are coronal, axial, or sagittal images.

In some embodiments, the method comprises obtaining organ distribution data for an amyloid-reactive agent or detection dye. In some embodiments, organ distribution data are images. The images produced using the imaging procedure embodied in the present invention may be analyzed by any method known in the art. For example, in some embodiments, imaging data derived from a PET or SPECT scan can be inputted into a processor that identifies individual pixels or groups of pixels whose brightness is greater than a predetermined threshold or an average background, and identified pixels may be characterized as indicating the presence of the amyloid-reactive reagent or dye. In another embodiment, the image data may be derived from images scanned and inputted into a processor. In such embodiments, a similar process that identifies bright spots on the image may be used to locate the amyloid-reactive reagent or dye in the image. In certain embodiments, the analysis of the image may further include determining the intensity, concentration, strength or combination thereof of the output brightness, which may be correlated to the amount of radiolabeled protein in the image, an area or region of the image, or a particular spot on the image. Without wishing to be bound by theory, an area or spot on an image having a greater intensity than other areas or spots may hold a higher concentration of radiolabeled protein targeted to, for example, an amyloid deposit, and thus may have a higher concentration of the radiolabeled-amyloid-reactive reagent or dye attached to the region where the amyloid-reactive reagent or dye localizes.

In some embodiments, the method for diagnosing a type of amyloid disease comprises analyzing images by the spatial location of regions of interest to which the administered amyloid-reactive agent or detectable dye are targeted. In other embodiments, analysis of the pharmacokinetics of the administered amyloid-reactive reagent or dye may provide information on the appropriate timing of injection of the amyloid-reactive reagent or dye. By identifying areas, regions, or spots on an image that correlate to the presence of a radiolabeled protein, the presence or absence of amyloids may be determined. For example, in some embodiments, identifying regions or spots where the amyloid-reactive agent or detection dye concentrates indicates the presence of amyloids. In some embodiments, images that correlate to the presence of an amyloid-reactive agent or detection dye are used to diagnose an amyloid disease in an individual.

In some embodiments, the method further comprises providing a diagnosis of a type of amyloid disease based upon the organ distribution pattern. In some embodiments, a particular organ distribution pattern is indicative of a particular type of amyloid disease. For example, in some embodiments, the heart to spleen, heart to liver, spleen to liver, spleen to kidney, kidney to heart, or kidney to liver ratio is used to diagnose ATTR. In some embodiments, the heart to spleen, heart to liver, spleen to liver, spleen to kidney, kidney to heart, or kidney to liver ratio is used to diagnose ALECT2. In some embodiments, the heart to spleen, heart to liver, spleen to liver, spleen to kidney, kidney to heart, or kidney to liver ratio is used to diagnose AL.

In some embodiments, different types of amyloid diseases have different relative organ to organ ratios. In some embodiments, one particular type of amyloid disease may have a higher liver to heart ratio than another. In some embodiments, the heart to spleen ratio for individuals diagnosed with AATR is higher than the heart to spleen ratio for individuals diagnosed with AL. In some embodiments, the heart to spleen ratio for individuals diagnosed with ATTR is higher than the heart to spleen ratio for individuals diagnosed with ALECT2. In some embodiments, if the heart to spleen ratio is above 1, the individual is diagnosed with ATTR. In some embodiments if the heart to spleen ratio is above 1.2, above 1.3, above 1.4 or above 1.5, the individual is diagnosed with ATTR. In some embodiments, if the heart to spleen ratio is below 1.5, the individual is diagnosed with AL or ALECT2. In some embodiments if the heart to spleen ratio is below 1.4, below 1.3, below 1.3, or below 1, the individual is diagnosed with AL or ALECT2.

In some embodiments, the heart to kidney ratio is used to diagnose a type of amyloid disease. In some embodiments, the heart to kidney ratio is higher in individuals diagnosed with ATTR than the heart to kidney ratio in individuals diagnosed with AL. In some embodiments, the heart to kidney ratio is higher in individuals diagnosed with ALECT2. In some embodiments, if the heart to kidney ratio is above 1, the individual is diagnosed with ATTR. In some embodiments, if the heart to kidney ratio is above 1.2, above 1.3, above 1.4 above 1.5, above 1.6, or above 1.8 the individual is diagnosed with ATTR. In some embodiments, if the heart to kidney ratio is below 1.8, the individual is diagnosed with AL or ALECT2. In some embodiments if the heart to kidney ratio is below 1.6, below 1.5, below 1.4, or below 1, the individual is diagnosed with AL or ALECT2.

In some embodiments, the heart to liver ratio is used to diagnose a type of amyloid disease. In some embodiments, the heart to liver ratio is higher in individuals diagnosed with ATTR than the heart to kidney ratio in individuals diagnosed with AL. In some embodiments, the heart to liver ratio is higher in individuals diagnosed with ALECT2. In some embodiments, if the heart to liver ratio is above 1, the individual is diagnosed with ATTR. In some embodiments, if the heart to liver ratio is above 1.6, above 1.8, above 2.0, above 2.2, or above 2.3, the individual is diagnosed with ATTR. In some embodiments, if the heart to liver ratio is below 2.3, the individual is diagnosed with AL or ALECT2. In some embodiments if the heart to liver ratio is below 2.2, below 2.0, below 1.8, or below 1.6, the individual is diagnosed with AL or ALECT2.

In some embodiments, the liver to spleen ratio is used to diagnose a type of amyloid disease. In some embodiments, if the liver to spleen ratio is above 0.7, the individual is diagnosed with ATTR. In some embodiments, if the liver to spleen ratio is above 0.8, above 0.9, above 1.0, or above 1.2, the individual is diagnosed with ATTR. In some embodiments, if the liver to spleen ratio is below 1.2, the individual is diagnosed with AL or ALECT2. In some embodiments if the heart to liver ratio is below 0.9, below 0.8, or below 1.7, the individual is diagnosed with AL or ALECT2.

In some embodiments, if the liver to heart ratio is above 0.3, the individual is diagnosed with AL. In some embodiments, if the liver to heart ratio is above 0.4, above 0.5, above 0.6, or above 0.7 the individual is diagnosed with AL. In some embodiments, if the liver to heart ratio is below 0.3 the individual is diagnosed with ATTR or ALECT2. In some embodiments, if the liver to heart ratio is below 0.4, below 0.6, or below 0.7, the individual is diagnosed with ATTR or ALECT2.

In some embodiments, if the spleen to heart ratio is above 0.5, the individual is diagnosed with AL. In some embodiments, if the spleen to heart ratio is above 0.6, above 0.7, or above 0.8, the individual is diagnosed with AL. In some embodiments, if the spleen to heart ratio is below 0.5, the individual is diagnosed with ATTR or ALECT2. In some embodiments, if the spleen to heart ratio is below 0.6, below 0.7, or below 0.8, the individual is diagnosed with ATTR or ALECT2.

In some embodiments, if the spleen to liver ratio is greater than 9, the individual is diagnosed with AL. In some embodiments, if the spleen to liver ratio is above 10, above 11, or above 12, the individual is diagnosed with AL. In some embodiments, if the spleen to liver is less than 9, the individual is diagnosed with ATTR or ALECT2. In some embodiments, if the spleen to liver ratio is less than 10, less than 11, or less than 12, the individual is diagnosed with ATTR or ALECT2.

In some embodiments, if the kidney to heart ratio is above than 0.5 the individual is diagnosed with AL. In some embodiments, if the kidney to heart ratio is above 0.6, above 0.7, or above 0.8, the individual is diagnosed with AL. In some embodiments, if the kidney to heart ratio is less than 0.5, the individual is diagnosed with ATTR or ALECT2. In some embodiments, if the kidney to heart ratio is less than 0.6, less than 0.7, or less than 0.8, the individual is diagnosed with ATTR or ALECT2.

In some embodiments, the organ to organ ratio is based upon a standard uptake value ratio (SUVR). In some embodiments, a SUVR is calculated using a blood pool as a reference tissue. In some embodiments, the SUVR is calculated for each organ by dividing the amount of amyloid detection agent or dye in the organ by the blood pool ratio. In some embodiments, the blood pool is a vein or artery. In some embodiments, the blood pool is the lumen of the thoracic aorta.

In some embodiments, the level amyloid-reactive agent or detection dye in the heart is highest in individuals with ATTR. In some embodiments, the level of amyloid-reactive agent or detection dye in the liver is highest in individuals with AL. In some embodiments, the level of amyloid-reactive agent or detection dye is highest in the spleen in individuals with ALECT2. In some embodiments, the level of amyloid reactive agent or detection dye is highest in the kidney in individuals with ALECT2.

In some embodiments, the level of amyloid reactive agent or detection dye is lowest in the heart in individuals with ALECT2. In some embodiments, the level of amyloid-reactive agent or detection dye in the liver is lowest in individuals with ATTR. In some embodiments, the level of amyloid-reactive agent or detection dye in the spleen is lowest in individuals with ATTR. In some embodiments, the level of amyloid-reactive agent or detection dye in the kidney is lowest in individuals with ATTR.

In some embodiments, the cutoff value for diagnosing a particular type of amyloid disease is selected based upon a certain p value. In some embodiments, the cutoff is selected to provide a p-value of less than 0.1, less than 0.05, less than 0.01, less than 0.005, or less than 0.001.

In some embodiments, the cutoff value for diagnosing a particular type of amyloid disease is selected based upon a desired sensitivity. In some embodiments, the cutoff is selected to provide a sensitivity of at least 60%, at least 70%, at least 75%, at least 80%, at least 85%, at least 90% or at least 95%.

In some embodiments, the cutoff value is selected based upon a desired specificity (i.e. the ability to differentiate between different types of amyloid diseases). In some embodiments, the cutoff is selected to provide a specificity of at least 60%, at least 70%, at least 75%, at least 80%, at least 85%, at least 90% or at least 95%.

One of ordinary skill in the art will appreciate that each of the ratios discussed herein can easily converted to its inverse. For example, a heart to spleen ratio of 2:1 (2) is the same as a spleen to heart ratio of 1:2 (0.5).

### II. Methods of Treatment

Some aspects of the present invention provide methods of treating an amyloid disease based upon an organ distribution pattern of an amyloid reactive agent or detection dye. In some embodiments, provided herein is a method of treating an amyloid disease comprising administering an amyloid-reactive agent or detection dye, measuring the organ distribution of the amyloid-reactive agent or detection dye, and selecting a treatment based upon the type of disease.

In some embodiments, the methods of treating an amyloid disease comprising administering an amyloid-reactive agent or detection dye, and measuring the organ distribution pattern of the amyloid-reactive agent or detection dye for one or more organs. In some embodiments, the organ distribution pattern of the amyloid-reactive agent or dye indicates a type of amyloid disease. In some embodiments, the methods further comprise selecting a treatment based upon the type of amyloid disease.

In some embodiments, the method comprises obtaining an organ distribution pattern of an amyloid-reactive agent or detection dye, wherein the organ distribution pattern indicates a particular type of amyloid disease, and administering a treatment based upon the amyloid disease.

In some embodiments, the methods of treating an amyloid disease comprise administering an amyloid-reactive agent or detection dye comprising an amyloid-reactive peptide. In some embodiments, the amyloid-reactive agent or detection dye comprises a peptide, a fusion protein, a small molecule compound, or an antibody or fragment.

In some embodiments, the methods of treating an amyloid disease comprise administering an amyloid-reactive agent or detection dye comprising an amyloid-reactive peptide. In some embodiments, the amyloid-reactive peptide is a peptide with amino acid sequence set forth as any one of SEQ ID NOS: 1-14. In some embodiments, the methods of treating an amyloid disease comprise an amyloid-reactive peptide with an amino acid sequence as set forth in SEQ ID NO: 13.

In some embodiments, the methods of treating an amyloid disease comprise administering an amyloid-reactive agent or detection dye comprising a detectable label to determine an organ distribution pattern. In some embodiments, the amyloid-reactive agent or detection dye comprises a fluorescent label, chemiluminescent tag, or a radiolabel. In some embodiments, the amyloid-reactive agent or detection dye comprises a radiolabel. In some embodiments, the radiolabeled amyloid-reactive agent or detection dye is ¹²⁴I-p5+14. In other embodiments, the radiolabeled amyloid-reactive agent or detection dye is florbetapir, flortaben, or flutemetamol. In some embodiments, the methods of treating an amyloid disease comprise p5+14. In some embodiments, the amyloid reactive agent is radiolabeled. In some embodiments, the radiolabel is ¹¹C, ¹⁸F, ¹¹¹In, ^{99m}Tc, and ¹²³I, ¹²⁴I, or ¹²⁵I. In some embodiments, the radiolabelled amyloid-reactive agent or detection dye is a radiolabeled amyloid-reactive peptide. In some embodiments, the radiolabeled amyloid-reactive peptide is a ¹²⁴I-labelled amyloid-reactive peptide. In other embodiments, the method for diagnosing a type of amyloid disease comprise administering ¹²⁴I-p5+14. In other embodiments, the radiolabeled amyloid-reactive agent or detection dye is florbetapir, flortaben, or flutemetamol.

In certain other embodiments, the amyloid-reactive agent or detection dye comprises a fluorescent label. In some embodiments, the fluorescently-labelled amyloid-reactive agent or detection dye is ThT. In some embodiments, the amyloid-reactive agent or detection dye is administered parenterally, paracancerally, transmucosally, tansdermally, intramuscularly, intravenously, intradermally, subcutaneously, intraperitonealy, intraventricularly, or intracranially. In some instances, the amyloid-reactive agent or detection dye is administered intravenously or intraperitonealy.

In some embodiments, the methods of treating an amyloid disease measuring the organ the organ distribution pattern of the amyloid-reactive agent or detection dye for one or more organs. In some embodiments, the methods for treating the type of amyloid disease comprise measuring the organ distribution pattern of the amyloid-reactive agent or detection dye in one or more of heart, spleen, liver, kidneys, peripheral nerves, the gastrointestinal tract, muscle, lungs, brain, and lymph nodes. In some embodiments, the one or more organs are abdominothoracic organs. In some embodiments, the one or more organs are heart, spleen, liver, or kidney. In some embodiments, the step of measuring the organ distribution pattern of the amyloid-reactive agent or detection dye in one or more organ comprise determining an organ uptake value for each organ. In some embodiments, the organ uptake value is a standard uptake value for each organ. In some embodiments, the standard uptake value is determined as the ratio of the amount of amyloid-reactive agent or detection dye detected in an organ, and the amount of amyloid-reactive agent or detection dye detected in blood. In some embodiments, the organ uptake value is indicative of the organ distribution pattern of the amyloid-reactive agent or detection dye.

In other embodiments, the step of measuring the organ distribution pattern of the amyloid-reactive agent or detection dye in one or more organ comprises calculating an organ-to-organ ratio for two or more organs. In some embodiments, the step of calculating an organ-to-organ ratio for two or more organs comprises calculating the ratio between the organ uptake value for a first organ and the organ uptake value for a second organ. In some instances, the organ-to-organ ration is selected from the group consisting of liver-to-heart, spleen-to-heart, spleen-to-liver, spleen-to-kidney, kidney-to-heart, and kidney-to-liver. In some instances, the organ-to-organ ratio is the heart-to-spleen ratio. In some embodiments, the ratio is the inverse of any of these ratios.

In some embodiments, the measuring the organ distribution pattern of the amyloid-reactive agent or detection dye comprises the analysis of imaging data generated by PET, CT, MRI, SPECT, PET/CT, PET/MRI, or other imaging techniques. In some embodiments, the step of measuring the organ distribution pattern of the amyloid-reactive reagent or detection dye comprises analysis of images by the spatial location of regions of interest.

In some embodiments, the organ distribution pattern indicates a type of amyloid disease. In some embodiments, the organ distribution pattern is used to select a particular treatment based upon a type of amyloid disease. In some embodiments, the method further comprises providing a diagnosis of a type of amyloid disease based upon the organ distribution pattern. In some embodiments, a particular organ distribution pattern is indicative of a particular type of amyloid disease. For example, in some embodiments, the heart to spleen, heart to liver, spleen to liver, spleen to kidney, kidney to heart, or kidney to liver ratio is used to diagnose ATTR. In some embodiments, the heart to spleen, heart to liver, spleen to liver, spleen to kidney, kidney to heart, or kidney to liver ratio is used to diagnose ALECT2. In some embodiments, the heart to spleen, heart to liver, spleen to liver, spleen to kidney, kidney to heart, or kidney to liver ratio is used to diagnose AL.

In some embodiments, the method comprises treating or selecting a treatment for a systemic amyloidosis. Some example amyloid diseases that can be diagnosed and/or treated with the methods disclosed herein include, but are not limited to, AA amyloidosis, AL amyloidosis, AH amyloidosis, A3 amyloidosis, ATTR amyloidosis, ALECT2 amyloidosis, and IAPP amyloidosis of type II diabetes, Alzheimer's disease, thyroid tumors, Parkinson's disease, a tauopathy, senile systemic amyloidosis, familial hemodialysis, senile systemic aging, aging pituitary disorder, iatrogenic syndrome, reactive chronic inflammation, thyroid tumors, myeloma or other forms of cancer. In some embodiments, the methods of treating an amyloid disease comprise selecting a treatment for a systemic amyloidosis. In some embodiments, the methods of treating an amyloid disease comprise selecting a treatment for AL amyloidosis, ATTR amyloidosis, or ALECT2 amyloidosis. In some embodiments, the treatment is a targeted therapy for an ATTR amyloidosis, an AL amyloidosis, or an ALECT2 amyloidosis.

In some embodiments, the treatment is a small molecule, an antibody, a peptide, a protein, a nucleic acid, and/or a gene therapy. In some embodiments the treatment is a targeted treatment that is specific a particular type of amyloid disease.

In some embodiments, the treatment is a targeted therapy for an ATTR amyloidosis, an AL amyloidosis, or an ALECT2 amyloidosis. In some embodiments, the treatment is a targeted therapy for ATTR amyloidosis. In some embodiments, the treatment comprises a TTR tetramer stabilizer. In some embodiments, the TTR tetramer stabilizer is epigallocatechin-3-gallate (EGCG), AG-10, CHF5074, tafadimis, or diflunisal. In some embodiments, the treatment comprises an antibody or fragment that binds misfolded TTR. In some embodiments, the antibody is PRX004. In some embodiments, the treatment comprises an oligonucleotide. In some embodiments, the oligonucleotide is a TTR silencer. In some embodiments, the TTR silencer is patisiran (ALN-TTR02), vutrisiran, inotersen, or AKCEA-TTR-LRx. In some embodiments, the treatment comprises an ATTR amyloid disruptor. In some embodiments, the treatment comprises doxycycline, tauroursodeoxycholic acid, or serum amyloid P (SAP). In some embodiments, the treatment comprises an organ transplant. In some embodiments, the treatment comprises a liver transplant.

In other embodiments, the treatment is a targeted therapy for AL amyloidosis. In some embodiments, the treatment comprises bortemozib, ixazomib, or cazilfomib. In some embodiments, the treatment comprises an antibody or fragment. In some embodiments, the treatment comprises daratumab, CAEL-101, elotuzumab, or belantamab mafodotin. In some embodiments, the treatment comprises a stem cell therapy. In some embodiments, the treatment comprises a corticosteroid. In some embodiments, the corticosteroid is dexamethasone.

In some embodiments, the method is used to eliminate a potential therapy for a patient having amyloidosis. In some embodiments, the method is used to diagnose one type of amyloidosis and eliminate therapies for other types of amyloidosis. In some embodiments, the method is used to diagnose ALECT2 and eliminate therapies for AL or ATTR amyloidosis.

In some embodiments, the method is used to differentiate types of amyloidosis in order to develop therapies specific for a specific type of amyloidosis. For example in some embodiments, the method is used to identify individuals with ALECT2 amyloidosis and develop a therapy specific to ALECT2 amyloidosis.

### III. Kits

Some aspects of the invention provide kits for diagnosing or detecting a type of amyloid disease in an individual with the methods described herein.

In some embodiments, the kit comprises an amyloid-reactive agent or detection dye and instructions for use. In some embodiments, the amyloid-reactive agent or detection dye comprising a detectable label. In some embodiments, the amyloid-reactive agent or detection dye is ¹²⁴I-p5+14. In other embodiments, the amyloid-reactive agent or detection dye is florbetapir, flortaben, or flutemetamol. In some embodiments, the amyloid-reactive agent or detection dye is ThT.

In some embodiments, the radiolabel is ¹¹C, ¹⁸F, ¹¹¹In, ^{99m}Tc, and ¹²³I, ¹²⁴I, or ¹²⁵I. In some embodiments, the radiolabelled amyloid-reactive agent or detection dye is a radiolabeled amyloid-reactive peptide. In some embodiments, the radiolabeled amyloid-reactive peptide is a ¹²⁴I-labelled amyloid-reactive peptide. In other embodiments, the method for diagnosing a type of amyloid disease comprise administering ¹²⁴I-p5+14. In other embodiments, the radiolabeled amyloid-reactive agent or detection dye is florbetapir, flortaben, or flutemetamol.

In some embodiments, the instructions comprises instructions for detecting the amyloid reactive agent or detection dye in one or more organs. In some embodiments the amyloid reactive agent or detection dye is detected in blood, heart, lungs, kidney, or spleen.

In some embodiments, the kit comprises instructions for calculating a SUVR ratio for one or more organs. In some embodiments, a SUVR is calculated using a blood pool as a reference tissue. In some embodiments, the SUVR is calculated for each organ by dividing the amount of amyloid detection agent or dye in the organ by the blood pool ratio. In some embodiments, the blood pool is a vein or artery. In some embodiments, the blood pool is the lumen of the thoracic aorta.

In some embodiments, the instructions comprise instructions for determining an organ to organ ratio. In some embodiments, the organ to organ ratio is the liver to heart, spleen to heart, spleen to liver, spleen to kidney, kidney to heart, kidney to liver, or the inverse of any of these ratios.

In some embodiments, the kit further comprises instructions for providing a diagnosis based upon the organ to organ ratio.

In some embodiments, the kit comprises a therapeutic agent for treating an amyloid disease.

### EXAMPLES

### Example 1—Quantification of ¹²⁴I-p5+14 Uptake in Organs of Patients Imaged Using PET/CT

This example describes the differentiation of amyloid type using data from PET images.

### Imaging of Amyloid Deposits in Patients

Positron emission tomography/x-ray computed tomography (PET/CT) images were obtained from light chain-associated (AL) amyloidosis, transytherin-associated (ATTR) amyloidosis and leukocyte chemotactic factor 2-associated (ALECT2) amyloidosis patients enrolled in the first 26-patient cohort of the a Phase 1/2 trial of ¹²⁴I-p5+14. The ¹²⁴I-p5+14 imaging agent is a radiolabeled amyloid-reactive polypeptide that can be used for imaging amyloid in subjects by PET/CT. The ¹²⁴I-p5+14 can be used to detect amyloid deposits in the heart, liver, spleen, and kidney, and data from PET/CT images obtained using ¹²⁴I-p5+14 can be readily quantified.

### Determination of Organ:Blood Uptake

PET images were analyzed manually using a region of interest (ROI) method. Using planar images, either coronal, axial or sagittal, the ROI was placed in the organ of interest using the CT data to guide accurate anatomic placement. Care was taken to avoid regions of the organ where major blood vessels are present. The ROI was large enough to encompass an average area of tissue, or it was focused on a specific region or anatomic area or interest. Data from the ROI was noted, and the mean radioactivity per unit volume was determined (Bq/cc).

A standard uptake value ratio (SUVR) was calculated using the blood pool as the reference tissue. For this study, the lumen of the thoracic aorta, immediately distal to the aortic arch, identified on the CT image served as the blood pool ROI. The radioactivity of a carefully placed blood pool ROI was determined (Bq/cc). The SUVR for each organ (heart, spleen, liver, and left kidney) was then calculated by dividing the tissue radioactivity by the blood pool radioactivity, yielding the organ-to-blood pool ratio (organ:blood pool ratio).

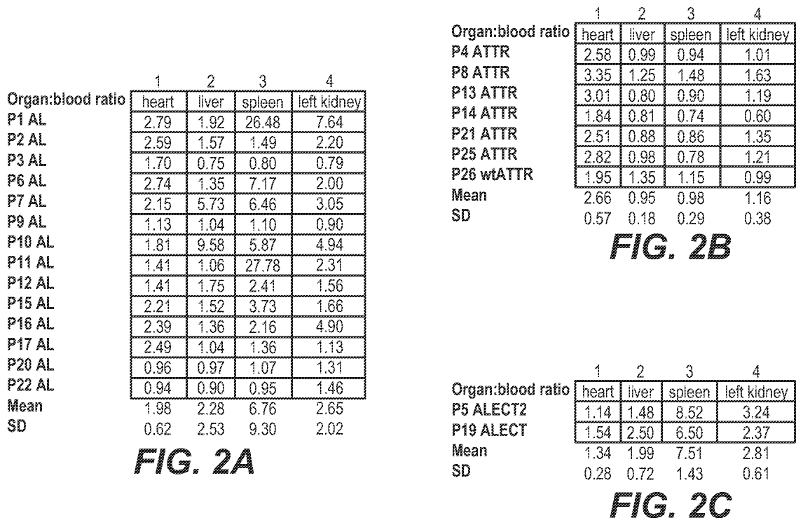

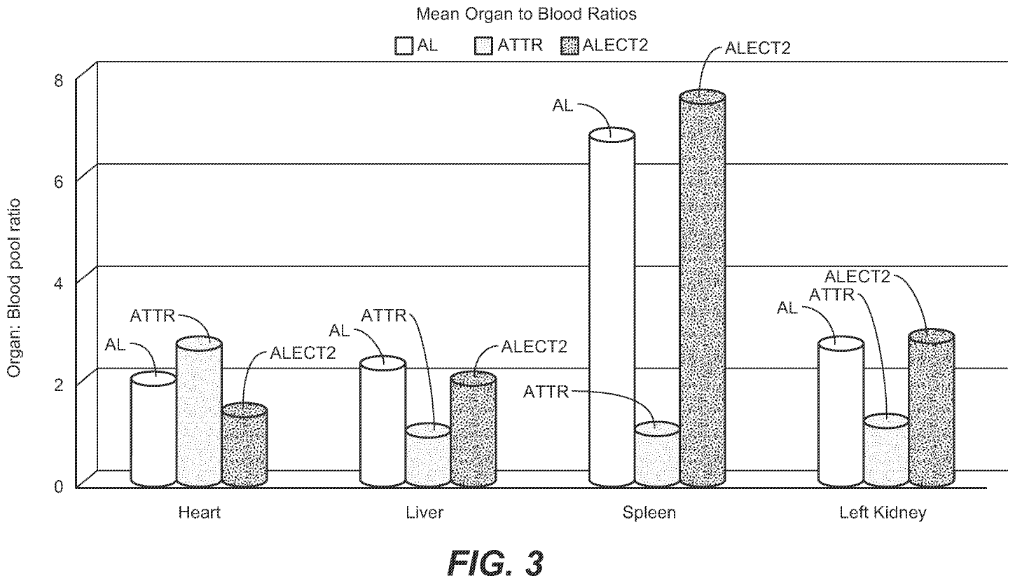

The organ:blood uptake was calculated for the AL, ATTR and ALECT2 patients enrolled in the first 26-patient cohort of the Phase 1/2 trial of ¹²⁴I-p5+14. The organ:blood uptake values for the patient cohort are summarized in FIG. 2. Analysis of the mean values for the entire populations yielded the relationships depicted in FIG. 3. The analysis revealed clear differences in the organ-specific uptake of ¹²⁴I-p5+14 for each of these patient cohorts, where:
Heart—ATTR>AL>ALECT2
Liver—AL>ALECT2>>ATTR
Spleen—ALECT2>AL>>ATTR
Kidney—ALECT2>AL>>ATTR

These relationships allowed for the development of an algorithm for differentiating AL from ATTR amyloidosis.

Differentiation of AL from ATTR

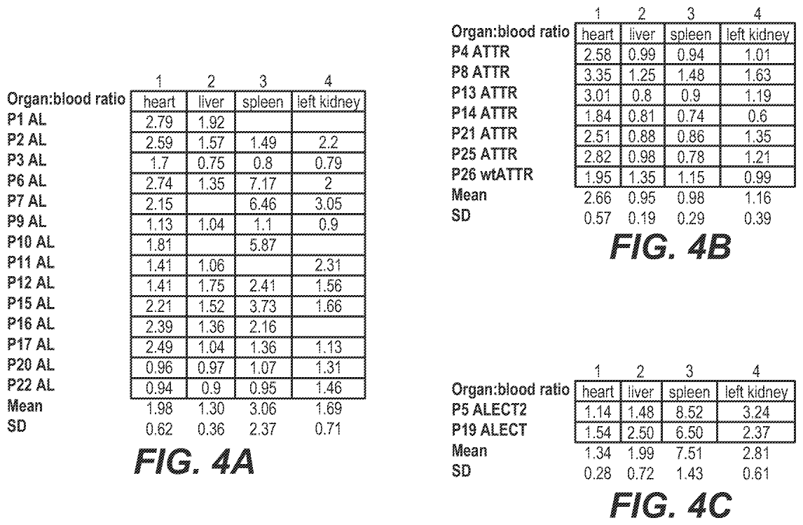

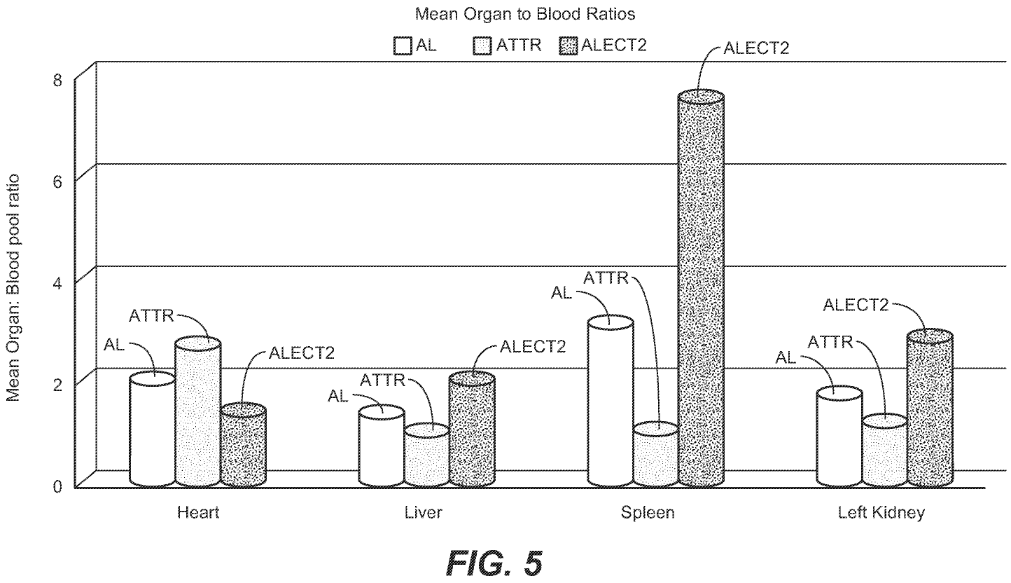

As a first step, the distribution for each organ was analyzed for normality using skewness and kurtosis metrics. Outliers that were 3.29 standard deviations above the mean, or higher, were then excluded in a list-wise fashion. FIG. 4 shows the dataset following exclusion of the outlier data points. Re-analysis of the mean values using the outlier-excluded data revealed the organ-specific relationships depicted in FIG. 5. After removal of the outliers from the population, the following relationships were observed:
Heart—ATTR>AL>ALECT2
Liver—ALECT2>ATTR>ATTR
Spleen—ALECT2>>AL>ATTR
Kidney—ALECT2>AL>ATTR

As a second step, the ability to differentiate AL from ATTR, and ATTR from AL was assessed by using receiver operator characteristic (ROC) analyses. ROC curves were generated for AL and ATTR using the SUVR values for individual organs and ratios of one organ to another (based initially on the data depicted in FIG. 5). The area under the curve (AUC) was determined, as was the significance and 95% confidence interval (95% CI) for the AUC. The sensitivity and specificity values for the optimal cutoff number that would differentiate AL from ATTR were also determined using the current data set.

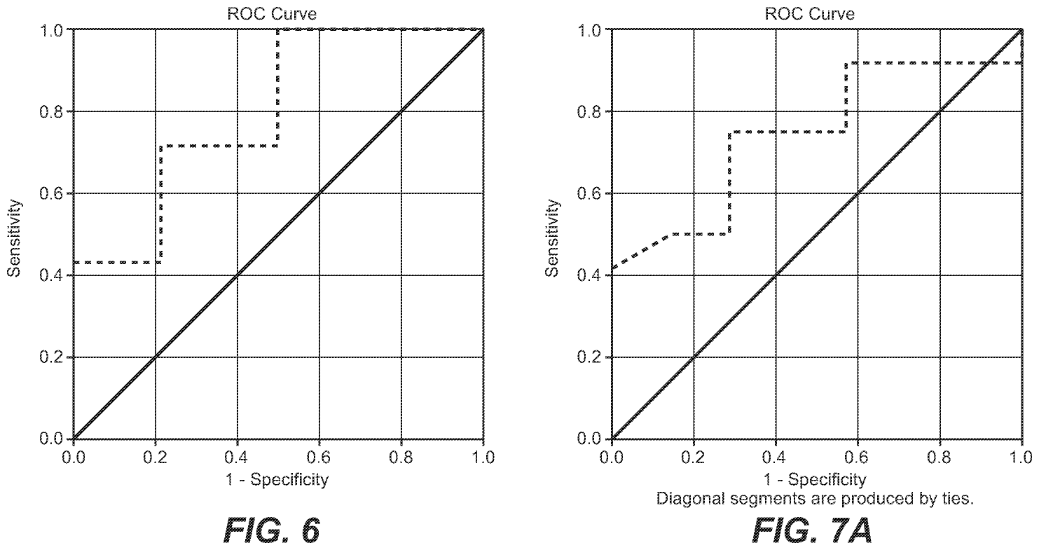

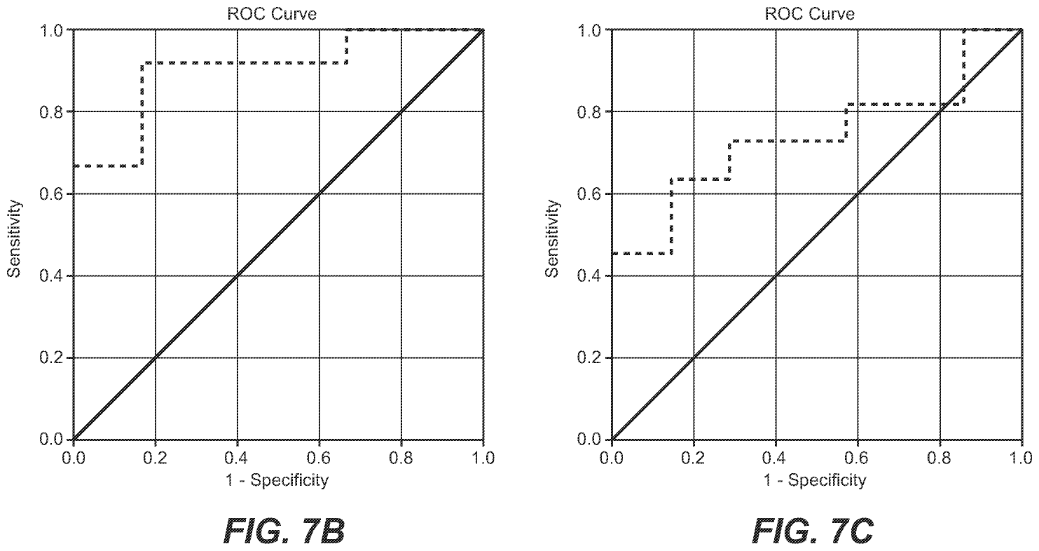

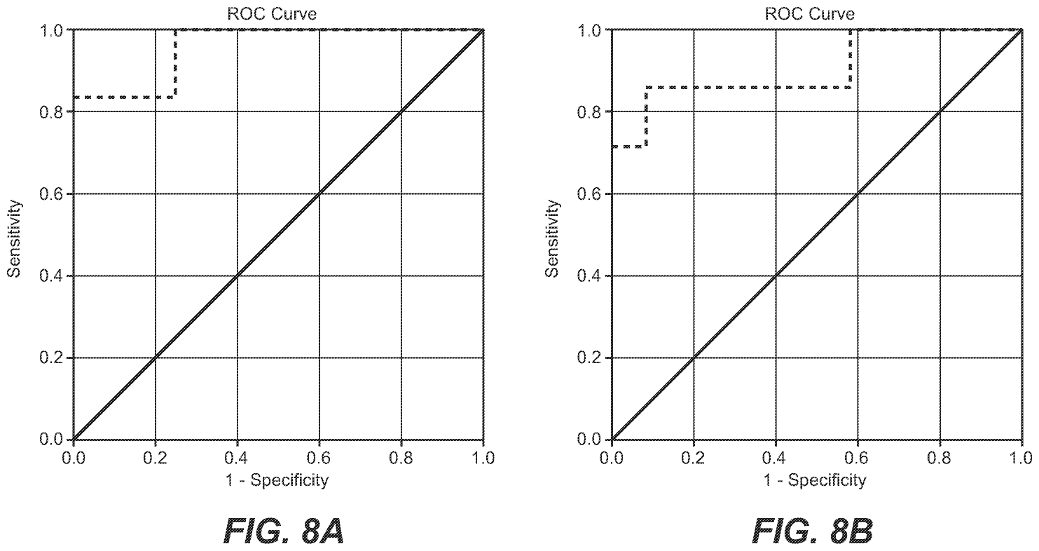

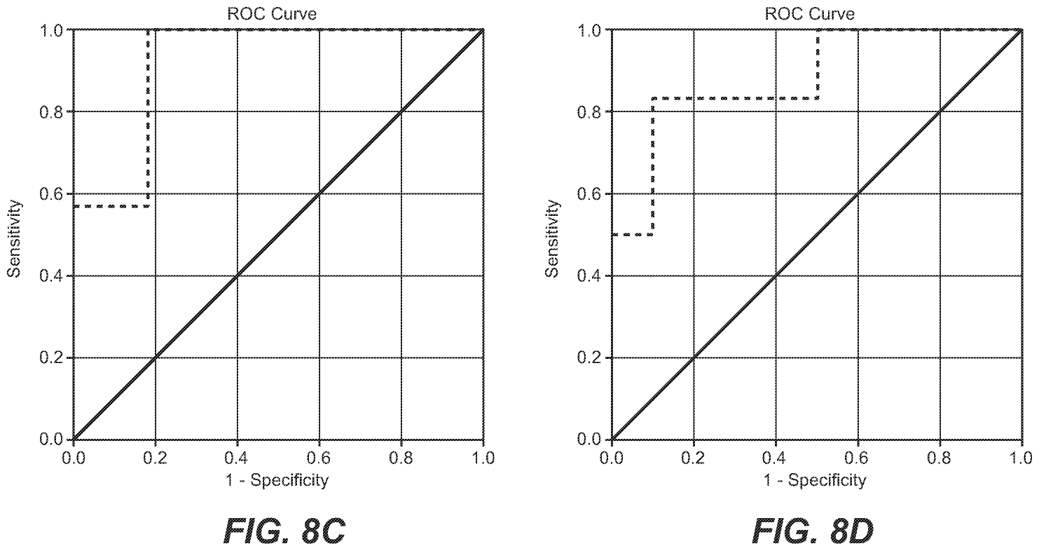

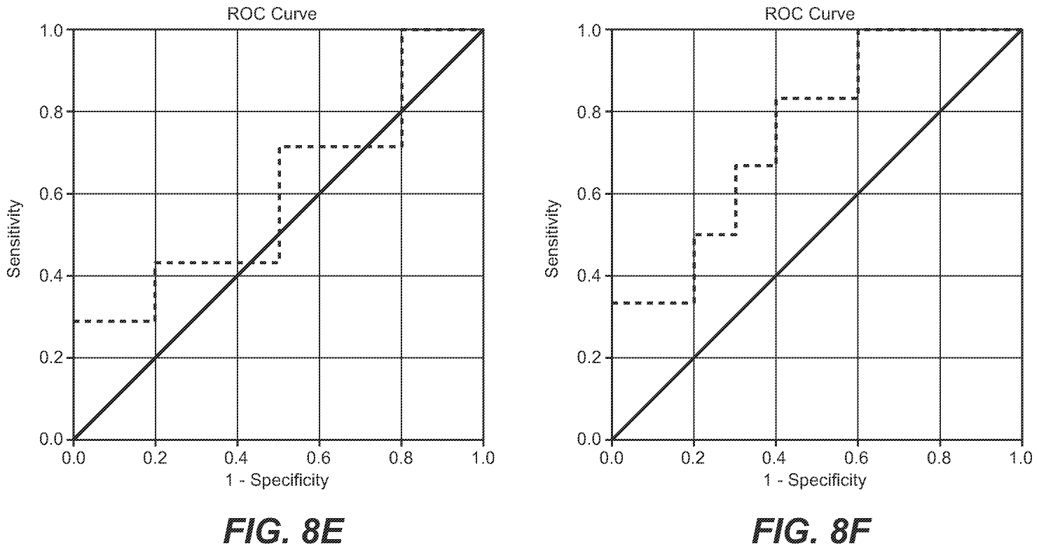

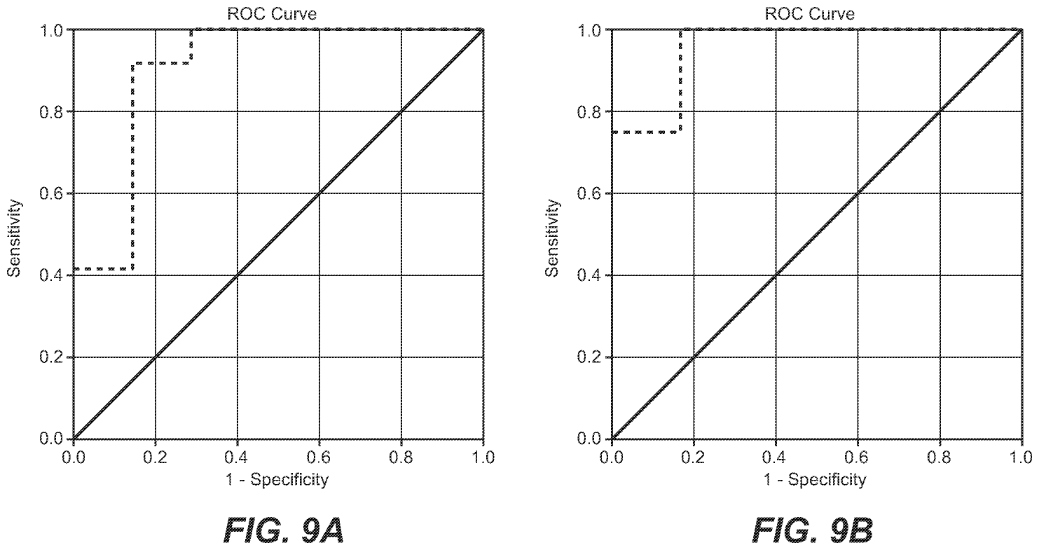

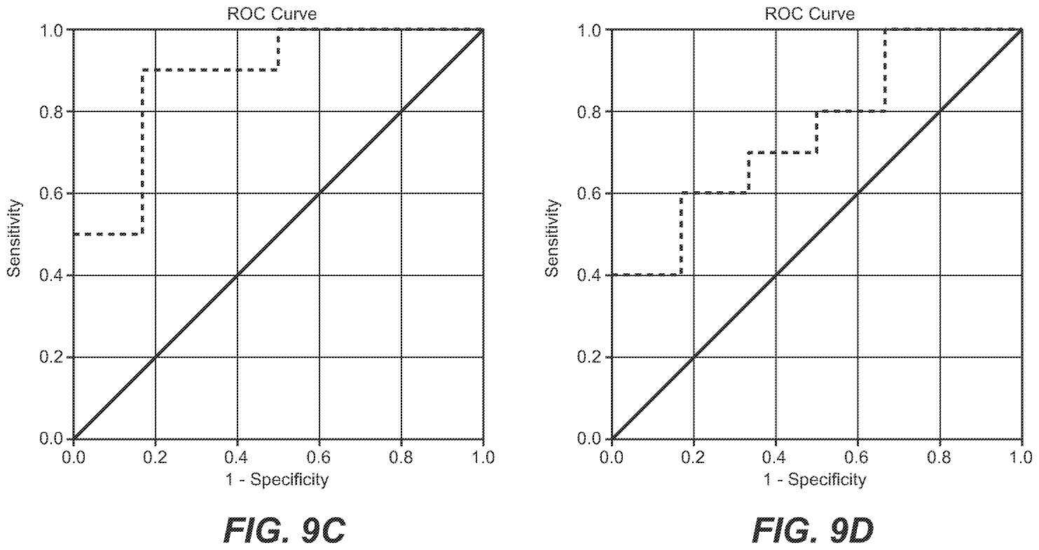

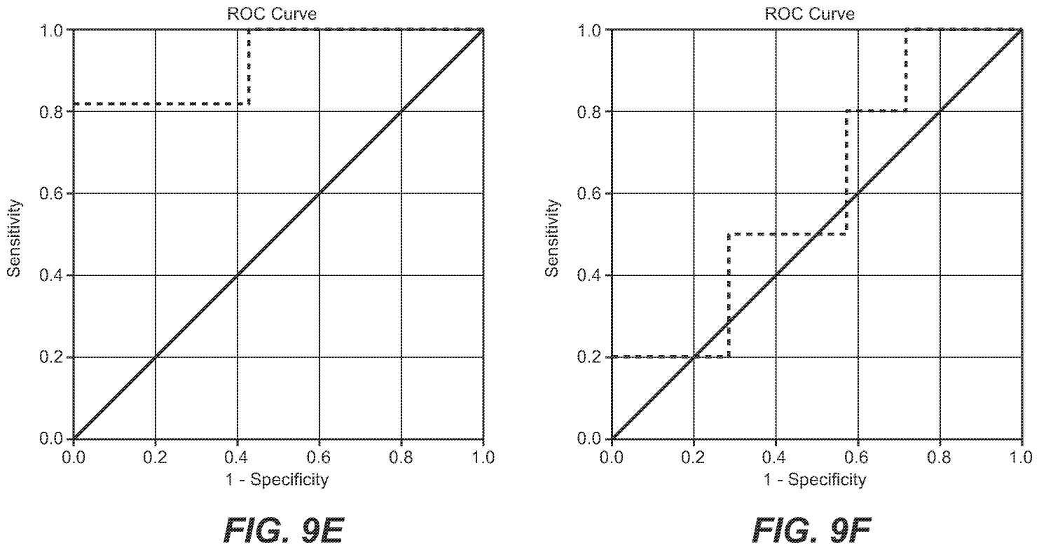

Table 2 summarizes the results of the ROC analysis for ATTR, while FIG. 6 shows the ROC curve for heart uptake and FIGS. 7A-7F show the ROC curves for organ-to-organ uptake ratios in ATTR. Table 3 summarizes the results of the ROC analysis for AL, while FIGS. 7A-7C show the ROC curves for individual organ uptake and FIGS. 8A-8F show the ROC curves for organ-to-organ uptake ratios in AL.

TABLE 2
Receiver Operator Characteristic (ROC) for detecting ATTR.

| Ratio | AUC | AUC 95% CI | p-value | Cut-off | Sensitivity | Specificity |
|---|---|---|---|---|---|---|
| Heart | 0.80 | 0.60-1.00 | 0.03 | 2.50 | 71.4% | 78.6% |
| Heart/Spleen | 0.96 | 0.87-1.00 | 0.002 | 1.40 | 100% | 75% |
| Heart/Liver | 0.91 | 0.74-1.00 | 0.004 | 2.27 | 85.7% | 91.7% |
| Heart/Kidney | 0.92 | 0.80-1.00 | 0.003 | 1.62 | 100% | 81.8% |
| Liver/Spleen | 0.88 | 0.71-1.00 | 0.013 | 0.99 | 83.3% | 90.0% |
| Liver/Kidney | 0.60 | 0.31-0.89 | 0.50 | — | — | — |
| Kidney/Spleen | 0.75 | 0.51-1.00 | 0.10 | — | — | — |

TABLE 3
Receiver Operator Characteristic (ROC) for detecting AL.

| Ratio | AUC | AUC 95% CI | p-value | Cut-off | Sensitivity | Specificity |
|---|---|---|---|---|---|---|
| Liver | 0.74 | 0.52-0.97 | 0.083 | — | — | — |
| Spleen | 0.90 | 0.76-1.00 | 0.007 | 1.01 | 83.3% | 83.3% |
| Kidney | 0.74 | 0.51-0.97 | 0.094 | — | — | — |
| Liver/Heart | 0.91 | 0.74-1.00 | 0.004 | 0.47 | 83.3% | 85.7% |
| Spleen/Heart | 0.96 | 0.87-1.00 | 0.002 | 0.56 | 83.3% | 83.3% |
| Spleen/Liver | 0.88 | 0.71-1.00 | 0.013 | 10.6 | 80.0% | 83.3% |
| Spleen/Kidney | 0.75 | 0.51-1.00 | 0.10 | — | — | — |
| Kidney/Heart | 0.92 | 0.80-1.00 | 0.003 | 0.63 | 81.1% | 100.0% |
| Kidney/Liver | 0.60 | 0.31-0.89 | 0.50 | — | — | — |

For the detection of ATTR patients, calculating the heart-to-spleen SUVR ratio provided the optimal method, with p=0.002 and an AUC of 0.96. Using a cutoff value of 1.40 yields a 100 sensitivity and 75% specificity for diagnosing ATTR. The heart-to-kidney SUVR ratio provided similarly good predictive values with marginally higher specificity.

The mean heart-to-spleen SUVR ratio for ATTR patients is higher than the heart-to-spleen SUVR ratio for AL patients. Additionally, based on the limited data for ALECT2 patients, this ratio is expected to be much lower in ALECT2 than in AL, suggesting that with more data points it would be possible to differentiate AL from ALECT2. Based on our current dataset for AL, ATTR and ALECT2 patients, using a one-way ANOVA with multiple comparisons the spleen SUVR of ALECT2 patients is significantly higher that both AL (p=0.02) and ATTR (p=0.002) patients.

The results of these analyses indicate that either the SUVR value for an individual organ or more precisely an organ-to-organ ratio, such as the heart-to-spleen SUVR ratio or heart-to-kidney SUVR ratio can be used to differentiate ATTR amyloidosis from AL amyloidosis, and potentially to differentiate ALECT2 amyloidosis from both AL and ATTR amyloidosis.

### CONCLUSIONS

Imaging data obtained was used to determine the heart amyloid amount-to-spleen amyloid amount, which can be used to discriminate between AL, ATTR and ALECT2 amyloids. As AL patients (as a population) have heart and spleen involvement, the heart amyloid amount-to-spleen amyloid amount ratio is about 1. ATTR patients have heart amyloid involvement but not spleen, so the ratio is greater than 1. ALECT2 patients have lots of splenic amyloid and very little (if any) heart amyloid so the ratio is much less than 1. Based on the current patient population, calculating the heart-to-spleen ratio can be used to determine, with some statistical level of certainty (e.g., >90%, >80%, etc.) which type of amyloid the patient has.

What is claimed is:

1. A method of diagnosing a type of amyloid disease comprising
administering a amyloid-reactive agent or detection dye to an individual; and
measuring an organ distribution pattern of the amyloid-reactive agent or detection dye in one or more organs of the individual,
wherein the organ distribution pattern of the amyloid-reactive agent or detection dye indicates a type of amyloid disease.

2. A method of treating an amyloid disease comprising
administering an amyloid-reactive agent or detection dye to an individual; and
measuring the organ distribution pattern of the amyloid-reactive agent or detection dye for one or more organs of the individual, wherein the organ distribution pattern of the amyloid-reactive agent or detection dye indicates a type of amyloid disease; and
selecting a treatment based upon the type of amyloid disease.

3. A method of diagnosing a type of amyloid disease comprising
receiving organ distribution pattern data for an amyloid-reactive-agent or detection dye for an individual; and
calculating an organ-to-organ ratio for two or more organs,
wherein the organ-to-organ ratio is used to diagnose a type of amyloid disease in the individual.

4. The method of any one of claims 1-3, wherein the type of amyloid disease comprises systemic amyloidosis.

5. The method of any one of claims 1-4, wherein the type of amyloid disease is selected from the group consisting of amyloid light chain amyloidosis (AL), transthyretin-associated amyloidosis (ATTR), and ALECT2.

6. The method of any one of claims 1-5, wherein the organ is selected from the group consisting of heart, spleen, kidney, and liver.

7. The method of anyone of claims 1-6, wherein the amyloid-reactive agent is an amyloid-reactive peptide that is detectably labeled.

8. The method of claim 7, wherein the amyloid-reactive peptide comprises the amino acid sequence set forth in SEQ ID NO: 1, SEQ ID NO: 2, SEQ ID NO: 13, or SEQ ID NO: 14.

9. The method of any one of claims 1-6, wherein the amyloid-reactive agent reacts with Aβ fibrils.

10. The method of any one of claims 1-6, wherein the amyloid-reactive agent reacts with synthetic fibrils composed of light chains or fragments thereof.

11. The method of any one of claims 1-6, wherein the amyloid-reactive agent is selected from the group consisting of florbetapir, florbetaben and flutemetamol.

12. The method of any one of claims 1-6, wherein the detection dye is ThT.

13. The method of any one of claims 1-12 wherein the organ distribution pattern is measured using PET/CT images.

14. The method of any one of claims 1-13, wherein the amyloid-reactive agent is radiolabeled.

15. The method of any one of claims 1-14, wherein an organ to blood ratio of the amyloid-reactive-agent or detection dye is calculated.

16. The method of any one of claims 1-15, wherein an organ to organ ratio of the amyloid-reactive-agent or detection dye is calculated.

17. The method of claim 16, wherein the organ to organ ratio is selected from the group consisting of liver to heart, spleen to heart, spleen to liver, spleen to kidney, kidney to heart, and kidney to liver.

18. The method of claim 17, wherein the organ to organ ratio is a heart to spleen ratio.

19. The method of claim 18, wherein if the heart to spleen ratio is above 1.4, the individual is diagnosed with ATTR amyloidosis.

20. The method of any one of claims 1-19, further comprising administering the treatment to the individual.

21. A kit for diagnosing a type of amyloid disease comprising an amyloid-reactive agent or detection dye and instructions for use.

22. The kit of claim 21, wherein the instructions for use comprises a method of diagnosis provided herein.

23. The kit of claim 21 or claim 22, wherein the kit is for detecting or diagnosing systemic amyloidosis.

24. The kit of any one of claims 21-23, wherein the kit is for detecting or diagnosing amyloid light chain amyloidosis (AL), transthyretin-associated amyloidosis (ATTR), or ALECT2.

25. The kit of any one of claims 21-24, wherein the amyloid-reactive agent is an amyloid-reactive peptide that is detectably labeled.

26. The method of claim 25, wherein the amyloid-reactive peptide comprises the amino acid sequence set forth in SEQ ID NO: 1, SEQ ID NO: 2, SEQ ID NO: 13, or SEQ ID NO: 14.

27. The kit of any one of claims 21-26, wherein the amyloid-reactive agent reacts with Aβ fibrils.

28. The kit of any one of claims 21-27, wherein the amyloid-reactive agent reacts with synthetic fibrils composed of light chains or fragments thereof.

29. The kit of any one of claims 21-28, wherein the amyloid-reactive agent is selected from the group consisting of florbetapir, florbetaben and flutemetamol.

30. The kit of any one of claims 21-29, wherein the detection dye is ThT.

31. The kit of any one of claims 21-30, comprising instructions for measuring an organ distribution pattern.

32. The kit of any one of claims 21-31, wherein the amyloid-reactive agent is radiolabeled.

33. The kit of any one of claims 21-32, comprising instructions for calculating an organ to blood ratio of the amyloid-reactive-agent or detection dye.

34. The kit of any one of claims 21-33, comprising instructions for calculating an organ to organ ratio of the amyloid-reactive-agent or detection dye.

35. The kit of claim 34, wherein the organ to organ ratio is selected from the group consisting of liver to heart, spleen to heart, spleen to liver, spleen to kidney, kidney to heart, and kidney to liver.

36. The kit of claim 35, comprising instructions to provide a diagnosis of ATTR amyloidosis if the heart to spleen ratio is above 1.4.

37. The kit of any one of claims 21-36 comprising instructions to administer a treatment to the individual based upon the diagnosis.

38. The kit of any one of claims 21-37, further comprising a therapeutic agent for treating a type of amyloid.
